(19) United States
(12) Patent Application Publication
(10) Pub. No.: US 2023/0059192 A1
(43) Pub. Date: Feb. 23, 2023

(54) CHARACTERIZING OLIGONUCLEOTIDES

(71) Applicant: CRISPR THERAPEUTICS AG, Zug (CH)

(72) Inventor: Luke J. Turechek, Cambridge, MA (US)

(21) Appl. No.: 17/821,018

(22) Filed: Aug. 19, 2022

Related U.S. Application Data
(60) Provisional application No. 63/234,885, filed on Aug. 19, 2021.

(51) Int. Cl. C12Q 1/6855; C12N 15/10; C12N 15/113

(52) U.S. Cl. CPC C12Q 1/6855; C12N 15/1065; C12N 15/113

(57) ABSTRACT

The present disclosure provides methods for determining oligonucleotide purity and/or characterizing small RNAs. The methods comprising ligating adapters comprising unique molecule identifiers (UMIs), amplifying ligation products to generate a library, and sequencing the library. The methods of the disclosure exhibit reduced or no bias in terms of discrepancies that can arise during the ligation and/or amplification steps of the methods.

### CROSS-REFERENCE TO RELATED APPLICATIONS

This application claims priority from U.S. Provisional Application No. 63/234,885 filed Aug. 19, 2021 the entire contents of which is hereby incorporated by reference.

### INCORPORATION BY REFERENCE OF SEQUENCE LISTING

This application contains a Sequence Listing that has been submitted in XML format via Patent Center and is hereby incorporated by reference in its entirety. The XML copy, created on Aug. 18, 2022, is named CT158-US_100867-737585_Sequence Listing.xml and is about 15,000 bytes in size.

### FIELD

This invention relates to methods for determining oligonucleotide purity and/or characterizing small RNAs.

### BACKGROUND

RNA-guided CRISPR-based systems have emerged as powerful genome modification tools due to their simplicity, target design plasticity, and multiplex targeting capacity. For example, a CRISPR-based system can comprise a CRISPR-based nuclease and a guide RNA (gRNA), which guides the CRISPR-based nuclease to a target site by base paring with the target site and interacting with the CRISPR-based nuclease. Algorithms are available for designing gRNAs with high on-target precision and low off-target effects, and gRNAs can readily be synthesized via in vitro transcription or phosphoramidite solid-phase synthesis. Once synthesized, however, there is no reliable method for determining the purity and quality of the gRNAs (or other synthetic RNA or DNA oligonucleotides). Mass spectrometry and high-performance liquid chromatography can be used to determine the purity of gRNAs but are not sensitive enough to detect the presence of undesirable sequences, such as gRNAs that do not have the intended number of nucleotides and/or the correct sequence, e.g., sequence variants. Traditional PCR based methods can be problematic because these methods can generate output that do not match the input or starting sequence either due to errors that crop up during amplification and/or to longer sequences being amplified less efficiently than shorter sequences. Accordingly, there is a need for alternative methods for accurately determining the purity and quality of the gRNAs (or other synthetic RNA or DNA oligonucleotides).

### SUMMARY

In some aspects, the present disclosure provides methods for characterizing oligonucleotides in a sample. A method comprises (a) providing a sample comprising a plurality of oligonucleotides, (b) ligating a plurality of 5′ adapters and 3′ adapters to the plurality of oligonucleotides to generate a plurality of adapter-ligated products, the 5′ adapter is ligated to the 5′ end of the oligonucleotide and the 3′ adapter is ligated to the 3′ end of the oligonucleotide, the 5′ adapter comprising a unique molecular identifier (UMI) comprising 5′ (N)₁₀₋₁₆RYRY(N)₁₋₅-3′, or optionally 5′-(N)₁₀₋₁₆ACAC(N)₁₋₅-3′, wherein N is A, C, G, or T/U, R is A or G, and Y is C or T/U, and the 3′ adapter comprising at least one random nucleotide at its 5′ end, or the 3′ adapter comprising a unique molecular identifier (UMI) and the 5′ adapter comprising at least one random nucleotide at its 3′ end, or both 3′ and 5′ adapter comprising unique molecular identifiers (UMI), (c) amplifying the plurality of adapter-ligated products using a forward primer and a reverse primer to generate a library, (d) sequencing the library to generate sequencing fragments of forward and reverse reads of the adapter-ligated products, wherein the amplified adapter-ligated product comprises the UMI, the oligonucleotide, and the 3′ adapter; and (e) analyzing and processing the sequencing fragments to determine the nucleotide sequences of the plurality of the oligonucleotides and the relative abundance of the nucleotide sequences thereby characterizing the oligonucleotides in the sample.

In some embodiments, the sequencing fragments are merged, counted, and binned based on the UMI, sequences with the same UMI sequences are deduplicated, the 5′ adapter sequences, which include the UMI sequences, and the 3′ adapter sequences are trimmed from the sequencing fragments thereby generating corresponding nucleotide sequences of the plurality of oligonucleotides, and the nucleotide sequences of the plurality of oligonucleotides are compared to a reference oligonucleotide sequence to identify full-length oligonucleotides with no sequence variation relative to the reference oligonucleotide. In some embodiments, the analyzing of the sequencing fragments further comprises identifying and quantifying sequence variants, wherein the sequence variants comprise 5′ truncated sequences, 3′ truncated sequences, sequences comprising a substitution, insertion, and/or deletion of at least one nucleotide, or a combination thereof.

In some embodiments, the 5′ adapter comprises 5′-(N)₁₀₋₁₆RYRY(N)₁₋₅-3′ and the sequencing fragments are filtered for the presence of the nucleotide sequence RYRY before trimming the 5′ adapter sequences, which include the UMI sequences, and the 3′ adapter sequences from the sequencing fragments. In some embodiments, the 5′ adapter comprises 5′-(N)₁₀₋₁₆ACAC(N)₁₋₅-3 and the sequencing fragments are filtered for the presence of the nucleotide sequence ACAC before trimming the 5′ adapter sequences, which include the UMI sequences, and the 3′ adapter sequences from the sequencing fragments. In some embodiments, the UMI of the 5′ adapter is 5′-N₁₃RYRYN-3′. In some embodiments, the UMI of the 5′ adapter is 5′-N₁₃ACACN-3′. In some embodiments, the UMI of the 5′ adapter is 5′-N₁₃RYRY(N)₁₋₅-3′. In some embodiments, the UMI of the 5′ adapter is 5′-N₁₃ACAC(N)₁₋₅-3′. In some embodiments, the 5′ adapters and 3′ adapters are ligated consecutively to the plurality of oligonucleotides. In some embodiments, the 3′ adapters are ligated to the plurality of oligonucleotides before the 5′ adapters. In some embodiments, the plurality of oligonucleotides is phosphorylated, and optionally purified by a chromatography method, prior to ligating the 3′ adapters. In some embodiments, the 3′ adapter further comprises a unique sequence at its 3′ end, the unique sequence comprising a complement sequence of a portion or all of a reverse primer. In some embodiments, the 5′ adapter further comprises a unique sequence located 5′ of the UMI, wherein the unique sequence corresponds to a portion or all of a forward primer. In general, the oligonucleotides are synthetic or naturally occurring. In some embodiments, oligonucleotides are gRNAs (e.g., sgRNAs or crRNAs), miRNAs, siRNAs, piRNAs, shRNAs, RNA adapters, RNA primers, RNA probes, antisense DNAs, DNA adapters, DNA primers, or DNA probes and their modified versions. In some embodiments, the oligonucleotides have a length of about 30-120 nucleotides. In some embodiments, the oligonucleotides are RNA.

In some embodiments, the method further comprises reverse transcribing the plurality of adapter-ligated products to generate a plurality of first strand cDNAs before step (c), wherein step (c) comprises synthesizing a plurality of second strand cDNAs from the plurality of first strand cDNAs and amplifying the plurality of first strand and second strand cDNAs in a first amplifying reaction to generate a preliminary library of amplified adapter-ligated products using a forward primer and a reverse barcode primer. In some aspects, the forward primer incorporates a 5′ sequencing adapter sequence, and optionally a barcode, to the 5′ end of the amplified adapter-ligated products, and the reverse barcode primer incorporates a 3′ sequencing adapter sequence, and optionally a barcode, to the 3′ end of the amplified adapter-ligated products, and the reverse barcode primer incorporates a barcode to the 3′ end of the amplified adapter-ligated products, wherein the barcode comprises 4 to 8 nucleotides, or optionally 6 nucleotides. In some instances, the method further comprises diluting the preliminary library to about 10,000-200,000 molecules and performing a second amplification reaction to generate a library of amplified adapter-ligated products using the forward primer and the reverse barcode primer.

In some embodiments, the 3′ adapter is DNA. In some embodiments, the 3′ adapter is pre-adenylated at its 5′ end and dideoxy-terminated at its 3′ end. In some embodiments, the 3′ adapter comprises two, three, four, or five random nucleotides (N) at its 5′ end. In some embodiments, the 3′ adapter comprises four random nucleotides (N) at its 5′ end. In some embodiments, the 3′ adapter comprises 5′-NNNNTGGAATTCTCGGGTGCCAAGGddC-3′ (SEQ ID NO: 1). In some embodiments, the 3′ adapter comprises 5′-rAppNNNNTGGAATTCTCGGGTGCCAAGGddC-3′ (SEQ ID NO: 2).

In some embodiments, the 5′ adapter is RNA and the UMI comprises 5′-(rN)₁₀₋₁₆rRrYrRrYr(N)₁₋₅-3′, or optionally 5′-(rN)₁₀₋₁₆rArCrArCr(N)₁₋₅-3′, wherein rN is rA, rC, rG, or rU, rR is rA or rG, and rY is rC or rU. In some instances, the UMI of the 5′ adapter is 5′-rN₁₃rRrYrRrYrN-3′. In some instances, the UMI of the 5′ adapter is 5′ -rN₁₃rArCrArCrN-3′. In some embodiments, the 5′ adapter comprises 5′-rGrUrUrCrArGrArGrUrUrCrUrArCrArGrUrCrCrGrArCrGrArUrCrN₁₃rArCrArCrN-3′ (SEQ ID NO: 3).

In some aspects, the present disclosure provides methods for characterizing small RNA in a sample. A method comprises (a) providing a sample comprising a plurality of small RNAs, (b) ligating a plurality of 3′ adapters to the plurality of small RNAs to generate a plurality of 3′ -ligated products, the 3′ adapter is ligated to the 3′ end of the small RNA, the 3′ adapter comprising at least one random nucleotide at its 5′ end and a unique sequence at its 3′ end, the unique sequence comprising a complement sequence of a portion or all of a reverse primer, (c) ligating a plurality of 5′ adapters to the plurality of 3′ -ligated products to generate a plurality of 5′ - and 3′-ligated products, the 5′ adapter is ligated to the 5′ end of the small RNA, the 5′ adapter comprising a unique molecular identifier (UMI) and a unique sequence located 5′ of the UMI, the UMI comprising 5′-(N)₁₀₋₁₆RYRY(N)₁₋₅-3′, or optionally 5′-(N)₁₀₋₁₆ACAC(N)₁₋₅-3′, wherein N is A, C, G, or T/U, R is A or G, and Y is C or T/U, and the unique sequence corresponding to a portion or all of a forward primer, (d) reverse transcribing the plurality of 5′- and 3′-ligated products with the reverse primer to generate a plurality of first strand cDNAs, (e) synthesizing a plurality of second strand cDNAs from the plurality of first strand cDNAs, optionally concurrently with step (f), (f) amplifying the plurality of first strand and second strand cDNAs in a first amplifying reaction to generate a preliminary library of amplified 5′- and 3′-ligated products using a forward primer and a reverse barcode primer, the forward primer incorporating a 5′ sequencing adapter sequence, and optionally a barcode, to the 5′ end of the amplified 5′- and 3′-ligated products, the reverse barcode primer incorporating a 3′ sequencing adapter sequence, and optionally a barcode, to the 3′ end of the amplified 5′- and 3′-ligated products, (g) diluting the preliminary library to about 10,000-200,000 molecules and performing a second amplification reaction to generate a library of amplified 5′- and 3′-ligated products using the forward primer and the reverse barcode primer, (h) sequencing the library to generate sequencing fragments of forward and reverse reads of the amplified 5′- and 3′-ligated products, wherein the amplified 5′- and 3′-ligated product comprises the UMI, the small RNA, and at least one random nucleotide, along with adaptor sequences and 1-2 barcodes, and (i) analyzing and processing the sequencing fragments to determine the nucleotide sequences of the plurality of the small RNAs and the relative abundance of the nucleotide sequences thereby characterizing the small RNAs in the sample.

In some embodiments, the sequencing fragments are merged, counted, and binned based on the UMI, sequences with the same UMI sequences are deduplicated, the 5′ adapter sequences, which include the UMI sequences, and the 3′ adapter sequences are trimmed from the sequence fragments thereby generating corresponding nucleotide sequences of the plurality of small RNAs, and the nucleotide sequences of the plurality of small RNAs are compared to a reference small RNA sequence to identify full-length small RNA with no sequence variation relative to the reference small RNA. In some embodiments, the analyzing of the sequencing fragments further comprises identifying and quantifying sequence variants, wherein the sequence variants comprise 5′ truncated sequences, 3′ truncated sequences, sequences comprising a substitution, insertion, and/or deletion of at least one nucleotide, or a combination thereof.

In some embodiments, the 5′ adapter comprises 5′-(N)₁₀₋₁₆RYRY(N)₁₋₅-3′ and the sequencing fragments are filtered for the presence of the nucleotide sequence RYRY before trimming the 5′ adapter sequences, which include the UMI sequences, and the 3′ adapter sequences from the sequencing fragments. In some embodiments, the 5′ adapter comprises 5′-(N)₁₀₋₁₆RYRY(N)₁₋₅-3′ and the sequencing fragments are filtered for the presence of the nucleotide sequence RYRY before trimming the 5′ adapter sequences, which include the UMI sequences, and the 3′ adapter sequences from the sequencing fragments.

In some embodiments, the small RNA is a synthetic RNA, the synthetic RNA being a gRNA (e.g., sgRNA, crRNA), a siRNA, a shRNA, an RNA adapter, an RNA primer, or an RNA probe. In some embodiments, the small RNA is a naturally occurring RNA, the naturally occurring RNA being a miRNA, a siRNA, or a piRNA. In some embodiments, the small RNA has a length from about 30-120 nucleotides. In some embodiments, the small RNA is phosphorylated, and optionally purified by a chromatography method, prior to step (b).

In some embodiments, the 3′ adapter is DNA. In some embodiments, the 3′ adapter is preadenylated at its 5′ end and dideoxy-terminated at its 3′ end. In some embodiments, the 3′ adapter comprises two, three, four, or five random nucleotides (N) at its 5′ end, wherein N is A, C, G, or T. In some embodiments, the 3′ adapter comprises 5′-NNNNTGGAATTCTCGGGTGCCAAGGddC-3′ (SEQ ID NO: 1). In some embodiments, the 3′ adapter comprises 5′-rAppNNNNTGGAATTCTCGGGTGCCAAGGddC-3′ (SEQ ID NO: 2). In some embodiments, the ligating in step (b) comprises contact with a T4 RNA ligase 2. In some embodiments, the 3′-ligated product is purified using magnetic beads prior to step (c).

In some embodiments, the 5′ adapter is RNA and the UMI comprises 5′-(rN)₁₀₋₁₆rRrYrRrYr(N)₁₋₅-3′, or optionally 5′-(rN)₁₀₋₁₆rArCrArCr(N)₁₋₅-3′, wherein rN is rA, rC, rG, or rU, rR is rA or rG, and rY is rC or rU. In some instances, the UMI of the 5′ adapter is 5′-rN₁₃rRrYrRrYrN-3′. In some instances, the UMI of the 5′ adapter is 5′ -rN₁₃rArCrArCrN-3′. In some embodiments, the 5′ adapter comprises 5′-rGrUrUrCrArGrArGrUrUrCrUrArCrArGrUrCrCrGrArCrGrArUrCrN₁₃rArCrArCrN-3′ (SEQ ID NO: 3). In some embodiments, the ligating in step (c) comprises contact with a T4 RNA ligase 1. In some embodiments, the 5′- and 3′-ligated product is purified using magnetic beads prior to step (d). In some embodiments, the first strand cDNA is purified using magnetic beads prior to step (e).

In some embodiments, the reverse barcode primer incorporates a barcode to the 3′ end of the amplified adapter-ligated products. In some embodiments, the forward primer also incorporates a barcode to the 5′ end of the amplified adapter-ligated products. In some embodiments, the barcode comprises 4 to 8 nucleotides, or optionally comprise 6 nucleotides. step (f) comprise about 3-6 amplification cycles, or optionally 5 amplification cycles. In some embodiments, the cDNA from step (f) is purified using magnetic beads prior to (g). In some embodiments, step (g) comprises about 30-34 amplification cycles, or optionally 32 amplification cycles. In some embodiments, the library is purified using magnetic beads prior to step (h). In some embodiments, the sequencing is a deep sequencing method.

Other features and advantages of this disclosure will become apparent in the following detailed description of embodiments of this invention, taken with reference to the accompanying drawings.

### BRIEF DESCRIPTION OF THE DRAWINGS

FIG. 1 presents the percentage of sequence purity of sgRNAs from two different suppliers (sgRNA-1A and sgRNA-2A from Supplier A and sgRNA-1B, sgRNA-2B, and sgRNA-3B from Supplier B).

FIG. 2 presents the percentages of sequence variants in sgRNA-1 from three different suppliers (Supplier A, Supplier B, and supplier C).

FIG. 3 presents the percentages of sequence variants in three replicates of sgRNA-1 from Supplier B.

FIG. 4 presents the percentages of sequence variants in sgRNA-2 from three different suppliers (Supplier A, Supplier B, and Supplier C).

FIG. 5 plots the percentage of measured versus expected 5′ truncated sgRNA (N-10) detected in full-length sgRNA spiked with different amounts of N-10.

### DETAILED DESCRIPTION

The present disclosure provides methods for characterizing oligonucleotides, e.g., synthetic RNA or DNA oligonucleotides and/or naturally occurring or cellular small RNAs. Such oligonucleotides can comprise mixtures of full-length intended or accurate sequences, truncated sequences, and/or sequences containing a substitution, insertion, and/or deletion. The disclosed methods allow the determination of the sequences of such oligonucleotides and the proportions that these oligonucleotides are present in the original sample. The methods of the disclosure comprise ligating 5′ and 3′ adapters to the oligonucleotide, amplifying the ligated product to create a library, sequencing the library, and analyzing the sequencing data to determine oligonucleotide purity and/or or characterize small RNA populations. The methods may also comprise adding sequencing adapters, and optionally barcodes, during the amplification process to create the library, diluting the library to generate a certain number of input or starting molecules, and re-amplifying the input or starting molecules to generate sufficient material for sequencing, i.e., sequencing fragments. The 5′ and 3′ adapters comprise random nucleotides (N) at the point of ligation to reduce ligation bias, and the 5′ adapter comprises an extended region of random nucleotides (N) to serve as unique molecular identifier (UMI) to reduce amplification bias. The sequencing fragments can be analyzed and processed to determine the nucleotide sequence of the oligonucleotides and/or the relative abundance of the nucleotide sequences.

The present disclosure provides a method for correcting amplification bias and sequencing errors by utilizing UMIs that have a randomized sequence and/or a filtering sequence. The method involves tagging each oligonucleotide in the sample with a unique sequence, i.e., ligating a 5′ adapter to each oligonucleotide in the sample. The 5′ adapter has a UMI that comprises a filtering sequence and at least 10-16 nucleotides that are randomly generated, allowing for up to 4¹⁰ to 4¹⁶ unique tags and increasing the likelihood that each oligonucleotide is tagged with a different UMI. The tagged oligonucleotides are subsequently amplified to generate populations of oligonucleotide sequences with the same “tag” that can be sequenced and binned and for which a consensus sequence can be generated. The filtering sequence allows for the identification of “good” UMIs and for “bad” or faulty UMIs to be discarded. The number of different UMIs with the same consensus sequence for the oligonucleotide correlates with the proportion of that nucleotide in the sample. Advantageously, the methods disclosed herein exhibited reduced or no bias in terms of discrepancies that can arise during the processing or manipulation of the oligonucleotides/small RNAs. For example, adapter ligation bias is minimized by including random nucleotides in both adapters at the point of ligation, and amplification bias is reduced by tagging each starting molecule with a unique sequence (UMI). In some aspects, the methods disclosed herein can be used to determine purity of synthetic oligonucleotides. In other aspects, the methods disclosed herein can be used to characterize and profile populations of small RNAs.

### I. METHODS FOR CHARACTERIZING OLIGONUCLEOTIDES

Provided herein are methods for characterizing oligonucleotides, e.g., oligonucleotide purity. The methods comprise (a) providing a sample comprising a plurality of oligonucleotides and (b) ligating a plurality of 5′ adapters and 3′ adapters to the plurality of oligonucleotides to generate a plurality of adapter-ligated products. The 5′ adapter comprises a unique molecular identifier (UMI) comprising 5′-(N)₁₀₋₁₆RYRY(N)₁₋₅-3′, or optionally 5′-(N)₁₀₋₁₆ACAC(N)₁₋₅-3′, wherein N is A, C, G, or T/U, R is A or G, and Y is C or T/U. The 3′ adapter comprises at least one random nucleotide at its 5′ end. The adapter-ligated products comprise the 5′ adapter comprising the UMI, the oligonucleotide, and the 3′ adapter. The methods further comprise (c) amplifying the plurality of adapter-ligated products using a forward primer and a reverse primer to generate a library; (d) sequencing the library to generate sequencing fragments of forward and reverse reads of the adapter-ligated products; and (e) analyzing and processing the sequencing fragments to determine the nucleotide sequences of the plurality of the oligonucleotides and the relative abundance of the nucleotide sequences thereby characterizing the oligonucleotides in the sample.

In some embodiments, the methods can comprise adding sequencing adapters, and optionally barcodes, to the adapter-ligated products during the amplification process to create the library, diluting the library to generate a certain number of input or starting molecules, and/or re-amplifying the input or starting molecules to generate sufficient material for sequencing. The analyzing can further comprise characterizing sequence variants and determining their relative abundance. In some embodiments, paired-end sequencing fragments are merged, counted, and binned based on the UMI. In some embodiments, sequences with the same UMI sequences are deduplicated, the 5′ adapter sequences, which include the UMI sequences, and the 3′ adapter sequences are trimmed from the sequencing fragments thereby generating corresponding nucleotide sequences of the plurality of oligonucleotides, and the nucleotide sequences of the plurality of oligonucleotides are compared to a reference oligonucleotide sequence to identify full-length oligonucleotides with no sequence variation relative to the reference oligonucleotide. In some embodiments, analyzing the sequencing fragments further comprises identifying and quantifying sequence variants. In some embodiments, the sequence variants comprise 5′ truncated sequences (i.e., a sequence that is missing one or more nucleotides at the 5′ end as compared to a reference sequence), 3′ truncated sequences (i.e., a sequence that is missing one or more nucleotides at the 3′ end as compared to a reference sequence), sequences comprising a substitution, insertion, and/or deletion of at least one nucleotide, or a combination thereof.

In some embodiments, the 5′ adapter comprises 5′-(N)₁₀₋₁₆RYRY(N)₁₋₅-3′ and the sequencing fragments are filtered for the presence of the nucleotide sequence RYRY before trimming the 5′ adapter sequences, which include the UMI sequences, and the 3′ adapter sequences from the sequencing fragments. In some embodiments, the 5′ adapter comprises 5′-(N)₁₀₋₁₆ACAC(N)₁₋₅-3 and the sequencing fragments are filtered for the presence of the nucleotide sequence ACAC before trimming the 5′ adapter sequences, which include the UMI sequences, and the 3′ adapter sequences from the sequencing fragments.

### Oligonucleotides

The methods of the disclosure can be used to determine the purity of a sample of oligonucleotides, e.g., a plurality of oligonucleotides. In general, the oligonucleotides can be synthetic oligonucleotides, which are generally prepared using phosphoramidite chemistry, or naturally occurring oligonucleotides. The plurality of oligonucleotides can comprise a mixture of full-length accurate sequences, 5′ truncated sequences, 3′ truncated sequences, and/or sequences comprising a substitution, insertion, and/or deletion of at least one nucleotide, or a mixture of thereof. The oligonucleotides can be RNA, DNA, or a mixture thereof.

Suitable oligonucleotides include gRNAs (e.g., sgRNAs, crRNAs), miRNAs, siRNAs,piRNAs, shRNAs, antisense RNA, RNA adapters, RNA primers, RNA probes, antisense DNA, DNA adapters, DNA primers, or DNA probes. A sgRNA comprises a 5′ spacer sequence and a 3′ sequence that forms a secondary structure and interacts with a CRISPR/Cas protein. A crRNA comprises a 5′ spacer sequence and a 3′ sequence, which base pairs with a tracrRNA.

The length of the oligonucleotide can and will vary depending upon its intended use. In general, the oligonucleotide can comprise from about 10 to about 250 nucleotides (nt). In some embodiments, the length of the oligonucleotide can range from about 15-120 nt. In various embodiments, the oligonucleotide can range in length from about 15-30 nt, from about 20-30 nt, from about 20-60 nt, from about 30-50 nt, from about 30-80 nt, from about 50-70 nt, from about 30-120 nt, from about 40-100 nt, from about 50-150 nt, from about 90-110 nt, from about 100-120 nt, from about 40-120 nt, from about 30-150, from about 20-200 nt, or from about 100-250 nt.

The oligonucleotide can comprise standard nucleobases, such adenine (A), guanine (G), thymine (T), cytosine (C), and uracil (U). In some embodiments, the oligonucleotide can comprise modified natural nucleobases, such as 5-methylcytosine (5meC), 5-(hydroxymethyl)cytosine (5hmC), 5-formylcytosine (5fC), 5-carboxycytosine (5caC), 5-(hydroxymethyl)uracil (5hmU), 5-formyluracil (5fU), dihydrouracil, pseudouracil, N⁶-methyladenine (5mA), xanthine, hypoxanthine, 7-methylguanine, and so forth. In embodiments in which the oligonucleotide is RNA, the oligonucleotide can comprise one or more substituted sugar moieties, e.g., one of the following at the 2′ position: OH, SH, SCH₃, F, OCN, OCH₃, OCH₃O(CH₂)ₙCH₃, O(CH₂).NH₂, or O(CH₂)ₙCH₃, where n is from 1 to about 10, alkyl or O-, S-, or N-alkyl, wherein alkyl is C₁ to C₁₀ alkyl. Similar modifications can be made at the 3′ position of the sugar on the 3′ terminal nucleotide of any oligonucleotide (e.g., RNA, DNA) and/or the 5′ position of the 5′ terminal nucleotide of any oligonucleotide (e.g., RNA, DNA). The oligonucleotide can comprise standard phosphodiester linkages and/or modified linkages such as phosphorothioates, phosphotriesters, morpholinos, methyl phosphonates, locked nucleic acids (LNA), peptide nucleic acids (PNA), short chain alkyl or cycloalkyl intersugar linkages, or short chain heteroatomic or heterocyclic intersugar linkages.

### Ligating Adapters

The disclosed methods involve providing a sample comprising a plurality of oligonucleotides and ligating a plurality of 5′ adapters and 3′ adapters to each end of the plurality of oligonucleotides to generate a plurality of adapter-ligated products, wherein the 5′ adapter is ligated to the 5′ end of the oligonucleotide and the 3′ adapter is ligated to the 3′ end of the oligonucleotide. The 5′ and 3′ adapters can be DNA, RNA, or a combination thereof. The 5′ and 3′ adapters can be single-stranded or double-stranded. In some embodiments, the 5′ adapter and the 3′ adapter are ligated sequentially to the oligonucleotide. In some embodiments, the 3′ adapter is ligated to the oligonucleotide before the 5′ adapter. In some embodiments, the 5′ adapter is ligated to the oligonucleotide before the 3′ adapter. In some embodiments, the 5′ adapter and the 3′ adapter are ligated concurrently to the oligonucleotide.

The 5′ adapter comprises a unique molecular identifier (UMI) comprising (5′-3′) about 10-16 random (N) nucleotides, about 3-5 semi-random nucleotides, and at least one random nucleotide. The random nucleotide at the 3′ end of the 5′ adapter (i.e., point of ligation) minimizes adapter ligation bias. In some embodiments, the UMI comprises (5′-3′) about 12-14 random nucleotides, about 3-5 semi-random nucleotides, and at least one random nucleotide. In specific embodiments, the UMI comprises (5′-3′) 13 random nucleotides, four semi-random nucleotides, and one random nucleotide. In some embodiments, a UMI of 18 nucleotides provides up to 4¹⁴ unique tags.

In some embodiments, the sequence of the UMI is 5′-(N)₁₀RYRYN-3′, wherein N is A, C, G, or T/U, R is A or G, and Y is C or T/U. In some embodiments, the sequence of the UMI is 5′-(N)₁₁RYRYN-3′. In some embodiments, the sequence of the UMI is 5′-(N)₁₂RYRYN-3′. In some embodiments, the sequence of the UMI is 5′-(N)₁₃RYRYN-3′. In some embodiments, the sequence of the UMI is 5′-(N)₁₄RYRYN-3′. In some embodiments, the sequence of the UMI is 5′-(N)₁₅RYRYN-3′. In some embodiments, the sequence of the UMI is 5′-(N)₁₆RYRYN-3′. In some embodiments, the sequence of the UMI is 5′-(N)₁₀RYRYNN-3′, wherein N is A, C, G, or T/U, R is A or G, and Y is C or T/U. In some embodiments, the sequence of the UMI is 5′-(N)₁₁RYRYNN-3′. In some embodiments, the sequence of the UMI is 5′-(N)₁₂RYRYNN-3′. In some embodiments, the sequence of the UMI is 5′-(N)₁₃RYRYNN-3′. In some embodiments, the sequence of the UMI is 5′-(N)₁₄RYRYNN-3′. In some embodiments, the sequence of the UMI is 5′-(N)₁₅RYRYNN-3′. In some embodiments, the sequence of the UMI is 5′-(N)₁₆RYRYNN-3′. In some embodiments, the sequence of the UMI is 5′-(N)₁₁RYRYNN-3′. In some embodiments, the sequence of the UMI is 5′-(N)₁₂RYRYNNN-3′. In some embodiments, the sequence of the UMI is 5′-(N)₁₃RYRYNNN-3′. In some embodiments, the sequence of the UMI is 5′-(N)₁₄RYRYNNN-3′. In some embodiments, the sequence of the UMI is 5′-(N)₁₅RYRYNNN-3′. In some embodiments, the sequence of the UMI is 5′-(N)₁₆RYRYNNN-3′. In some embodiments, the sequence of the UMI is 5′-(N)₁₁RYRYNNNN-3′. In some embodiments, the sequence of the UMI is 5′-(N)₁₂RYRYNNNN-3′. In some embodiments, the sequence of the UMI is 5′-(N)₁₃RYRYNNNN-3′. In some embodiments, the sequence of the UMI is 5′-(N)₁₄RYRYNNNN-3′. In some embodiments, the sequence of the UMI is 5′-(N)₁₅RYRYNNNN-3′. In some embodiments, the sequence of the UMI is 5′-(N)₁₆RYRYNNNN-3′. In some embodiments, the sequence of the UMI is 5′-(N)₁₁RYRYNNNNN-3′. In some embodiments, the sequence of the UMI is 5′-(N)₁₂RYRYNNNNN-3′. In some embodiments, the sequence of the UMI is 5′-(N)₁₃RYRYNNNNN-3′. In some embodiments, the sequence of the UMI is 5′-(N)₁₄RYRYNNNNN-3′. In some embodiments, the sequence of the UMI is 5′-(N)₁₅RYRYNNNNN-3′. In some embodiments, the sequence of the UMI is 5′-(N)₁₆RYRYNNNNN-3′. In some embodiments, the sequence of the UMI is 5′-(N)₁₀ACACN-3′. In some embodiments, the sequence of the UMI is 5′-(N)₁₁ACACN-3′. In some embodiments, the sequence of the UMI is 5′-(N)₁₂ACACN-3′. In some embodiments, the sequence of the UMI is 5′-(N)₁₃ACACN-3′. In some embodiments, the sequence of the UMI is 5′-(N)₁₄ACACN-3′. In some embodiments, the sequence of the UMI is 5′-(N)₁₅ACACN-3′. In some embodiments, the sequence of the UMI is 5′-(N)₁₆ACACN-3′. In some embodiments, the sequence of the UMI is 5′-(N)₁₀ACACNN-3′. In some embodiments, the sequence of the UMI is 5′-(N)₁₁ACACNN-3′. In some embodiments, the sequence of the UMI is 5′-(N)₁₂ACACNN-3′. In some embodiments, the sequence of the UMI is 5′-(N)₁₃ACACNN-3′. In some embodiments, the sequence of the UMI is 5′-(N)₁₄ACACNN-3′. In some embodiments, the sequence of the UMI is 5′-(N)₁₅ACACNN-3′. In some embodiments, the sequence of the UMI is 5′-(N)₁₆ACACNN-3′. In some embodiments, the sequence of the UMI is 5′-(N)₁₀ACACNNN-3′. In some embodiments, the sequence of the UMI is 5′-(N)₁₁ACACNNN-3′. In some embodiments, the sequence of the UMI is 5′-(N)₁₂ACACNNN-3′. In some embodiments, the sequence of the UMI is 5′-(N)₁₃ACACNNN-3′. In some embodiments, the sequence of the UMI is 5′-(N)₁₄ACACNNN-3′. In some embodiments, the sequence of the UMI is 5′-(N)₁₅ACACNNN-3′. In some embodiments, the sequence of the UMI is 5′-(N)₁₆ACACNNN-3′. In some embodiments, the sequence of the UMI is 5′(N)₁₀ACACNNNN-3′. In some embodiments, the sequence of the UMI is 5′-(N)₁₁ACACNNNN-3′. In some embodiments, the sequence of the UMI is 5′-(N)₁₂ACACNNNN-3′. In some embodiments, the sequence of the UMI is 5′-(N)₁₃ACACNNNN-3′. In some embodiments, the sequence of the UMI is 5′-(N)₁₄ACACNNNN-3′. In some embodiments, the sequence of the UMI is 5′-(N)₁₅ACACNNNN-3′. In some embodiments, the sequence of the UMI is 5′-(N)₁₆ACACNNNN-3′. In some embodiments, the sequence of the UMI is 5′-(N)₁₀ACACNNNNN-3′. In some embodiments, the sequence of the UMI is 5′-(N)₁₁ACACNNNNN-3′. In some embodiments, the sequence of the UMI is 5′-(N)₁₂ACACNNNNN-3′. In some embodiments, the sequence of the UMI is 5′-(N)₁₃ACACNNNNN-3′. In some embodiments, the sequence of the UMI is 5′-(N)₁₄ACACNNNNN-3′. In some embodiments, the sequence of the UMI is 5′-(N)₁₅ACACNNNNN-3′. In some embodiments, the sequence of the UMI is 5′-(N)₁₆ACACNNNNN-3′. In some embodiments, the sequence of the UMI is 5′-(N)₁₀ACACNNNNNN-3′. In some embodiments, the sequence of the UMI is 5′-(N)₁₁ACACNNNNNN-3′. In some embodiments, the sequence of the UMI is 5′-(N)₁₂ACACNNNNNN-3′. In some embodiments, the sequence of the UMI is 5′-(N)₁₃ACACNNNNNN-3′. In some embodiments, the sequence of the UMI is 5′-(N)₁₄ACACNNNNNN-3′. In some embodiments, the sequence of the UMI is 5′-(N)₁₅ACACNNNNNN-3′. In some embodiments, the sequence of the UMI is 5′-(N)₁₆ACACNNNNNN-3′. In particular embodiments, the sequence of the UMI is 5′-(N)₁₃RYRYN-3′. In specific embodiments, the sequence of the UMI is 5′-(N)₁₃ACACN-3′. In some embodiments, the sequence of the UMI is 5′-GTTCAGAGTTCTACAGTCCGACGATCNNNNNNNNNNNNNACAC-3′(SEQ ID NO:4). The 5∝ adapter further comprises a unique sequence located 5′ to the UMI, wherein the unique sequence corresponds to a portion or all of a forward primer used during the amplification step. The overall length of the 5′ adapter can range from about 30-70 nucleotides, from about 35-50 nucleotides, or from about 40-45 nucleotides.

In some embodiments, the 5′ adapter is RNA and the sequence of the UMI comprises 5′-(rN)₁₀rRrYrRrYrN-3′, wherein rN is rA, rC, rG, or rU, rR is rA or rG, and rY is rC or rU. In some embodiments, the sequence of the UMI of the 5′ adapter is 5′-(rN)₁₁rRrYrRrYrN-3′. In some embodiments, the sequence of the UMI of the 5′ adapter is 5′-(rN)₁₂rRrYrRrYrN-3′. In some embodiments, the sequence of the UMI of the 5′ adapter is 5′-(rN)₁₃rRrYrRrYrN-3′. In some embodiments, the sequence of the UMI of the 5′ adapter is 5′-(rN)₁₄rRrYrRrYrN-3′. In some embodiments, the sequence of the UMI of the 5′ adapter is 5′-(rN)₁₅rRrYrRrYrN-3′. In some embodiments, the sequence of the UMI of the 5′ adapter is 5′-(rN)₁₆rRrYrRrYrN-3′. In some embodiments, the sequence of the UMI of the 5′ adapter is 5′-(rN)₁₀rArCrArCrN-3′. In some embodiments, the sequence of the UMI of the 5′ adapter is 5′-(rN)₁₁rArCrArCrN-3′. In some embodiments, the sequence of the UMI of the 5′ adapter is 5′-(rN)₁₂rArCrArCrN-3′. In some embodiments, the sequence of the UMI of the 5′ adapter is 5′-(rN)₁₃rArCrArCrN-3′. In some embodiments, the sequence of the UMI of the 5′ adapter is 5′-(rN)₁₄rArCrArCrN-3′. In some embodiments, the sequence of the UMI of the 5′ adapter is 5′-(rN)₁₅rArCrArCrN-3′. In some embodiments, the UMI of the 5′ adapter is 5′-(rN)₁₆rArCrArCrN-3′. In some embodiments, the sequence of the UMI of the 5′ adapter is 5′-(rN)₁₀rRrYrRrYrNrN-3′. In some embodiments, the sequence of the UMI of the 5′ adapter is 5′-(rN)₁₁rRrYrRrYrNrN-3′. In some embodiments, the sequence of the UMI of the 5′ adapter is 5′-(rN)₁₂rRrYrRrYrNrN-3′. In some embodiments, the sequence of the UMI of the 5′ adapter is 5′-(rN)₁₃rRrYrRrYrNrN-3′. In some embodiments, the sequence of the UMI of the 5′ adapter is 5′-(rN)₁₄rRrYrRrYrNrN-3′. In some embodiments, the sequence of the UMI of the 5′ adapter is 5′-(rN)₁₅rRrYrRrYrNrN-3′. In some embodiments, the sequence of the UMI of the 5′ adapter is 5′-(rN)₁₆rRrYrRrYrNrN-3′. In some embodiments, the sequence of the UMI of the 5′ adapter is 5′-(rN)₁₀rArCrArCrNrN-3′. In some embodiments, the sequence of the UMI of the 5′ adapter is 5′-(rN)₁₁rArCrArCrNrN-3′. In some embodiments, the sequence of the UMI of the 5′ adapter is 5′-(rN)₁₂rArCrArCrNrN-3′. In some embodiments, the sequence of the UMI of the 5′ adapter is 5′-(rN)₁₃rArCrArCrNrN-3′. In some embodiments, the sequence of the UMI of the 5′ adapter is 5′-(rN)₁₄rArCrArCrNrN-3′. In some embodiments, the sequence of the UMI of the 5′ adapter is 5′-(rN)₁₅rArCrArCrNrN-3′. In some embodiments, the UMI of the 5′ adapter is 5′-(rN)₁₆rArCrArCrNrN-3′. In some embodiments, the sequence of the UMI of the 5′ adapter is 5′-(rN)₁₀rRrYrRrYrNrNrN-3′. In some embodiments, the sequence of the UMI of the 5′ adapter is 5′-(rN)₁₁rRrYrRrYrNrNrN-3′. In some embodiments, the sequence of the UMI of the 5′ adapter is 5′-(rN)₁₂rRrYrRrYrNrNrN-3′. In some embodiments, the sequence of the UMI of the 5′ adapter is 5′-(rN)₁₃rRrYrRrYrNrNrN-3′. In some embodiments, the sequence of the UMI of the 5′ adapter is 5′-(rN)₁₄rRrYrRrYrNrNrN-3′. In some embodiments, the sequence of the UMI of the 5′ adapter is 5′-(rN)₁₅rRrYrRrYrNrNrN-3′. In some embodiments, the sequence of the UMI of the 5′ adapter is 5′-(rN)₁₆rRrYrRrYrNrNrN-3′. In some embodiments, the sequence of the UMI of the 5′ adapter is 5′-(rN)₁₀rArCrArCrNrNrN-3′. In some embodiments, the sequence of the UMI of the 5′ adapter is 5′-(rN)₁₁rArCrArCrNrNrN-3′. In some embodiments, the sequence of the UMI of the 5′ adapter is 5′-(rN)₁₂rArCrArCrNrNrN-3′. In some embodiments, the sequence of the UMI of the 5′ adapter is 5′-(rN)₁₃rArCrArCrNrNrN-3′. In some embodiments, the sequence of the UMI of the 5′ adapter is 5′-(rN)₁₄rArCrArCrNrNrN-3′. In some embodiments, the sequence of the UMI of the 5′ adapter is 5′-(rN)₁₅rArCrArCrNrNrN-3′. In some embodiments, the UMI of the 5′ adapter is 5′-(rN)₁₆rArCrArCrNrNrN-3′. In some embodiments, the sequence of the UMI of the 5′ adapter is 5′-(rN)₁₀rRrYrRrYrNrNrNrN-3′. In some embodiments, the sequence of the UMI of the 5′ adapter is 5′-(rN)₁₁rRrYrRrYrNrNrNrN-3′. In some embodiments, the sequence of the UMI of the 5′ adapter is 5′-(rN)₁₂rRrYrRrYrNrNrNrN-3′. In some embodiments, the sequence of the UMI of the 5′ adapter is 5′-(rN)₁₃rRrYrRrYrNrNrNrN-3′. In some embodiments, the sequence of the UMI of the 5′ adapter is 5′-(rN)₁₄rRrYrRrYrNrNrNrN-3′. In some embodiments, the sequence of the UMI of the 5′ adapter is 5′-(rN)₁₅rRrYrRrYrNrNrNrN-3′. In some embodiments, the sequence of the UMI of the 5′ adapter is 5′-(rN)₁₆rRrYrRrYrNrNrNrN-3′. In some embodiments, the sequence of the UMI of the 5′ adapter is 5′-(rN)₁₀rArCrArCrNrNrNrN-3′. In some embodiments, the sequence of the UMI of the 5′ adapter is 5′-(rN)₁₁rArCrArCrNrNrNrN-3′. In some embodiments, the sequence of the UMI of the 5′ adapter is 5′-(rN)₁₂rArCrArCrNrNrNrN-3′. In some embodiments, the sequence of the UMI of the 5′ adapter is 5′-(rN)13rArCrArCrNrNrNrN-3′. In some embodiments, the sequence of the UMI of the 5′ adapter is 5′-(rN)₁₄rArCrArCrNrNrNrN-3′. In some embodiments, the sequence of the UMI of the 5′ adapter is 5′-(rN)₁₅rArCrArCrNrNrNrN-3′. In some embodiments, the UMI of the 5′ adapter is 5′-(rN)₁₆rArCrArCrNrNrNrN-3′. In some embodiments, the sequence of the UMI of the 5′ adapter is 5′-(rN)₁₀rRrYrRrYrNrNrNrNrN-3′. In some embodiments, the sequence of the UMI of the 5′ adapter is 5′-(rN)₁₁rRrYrRrYrNrNrNrNrN-3′. In some embodiments, the sequence of the UMI of the 5′ adapter is 5′-(rN)₁₂rRrYrRrYrNrNrNrNrN-3′. In some embodiments, the sequence of the UMI of the 5′ adapter is 5′-(rN)₁₃rRrYrRrYrNrNrNrNrN-3′. In some embodiments, the sequence of the UMI of the 5′ adapter is 5′-(rN)₁₄rRrYrRrYrNrNrNrNrN-3′. In some embodiments, the sequence of the UMI of the 5′ adapter is 5′-(rN)₁₅rRrYrRrYrNrNrNrNrN-3′. In some embodiments, the sequence of the UMI of the 5′ adapter is 5′-(rN)₁₆rRrYrRrYrNrNrNrNrN-3′. In some embodiments, the sequence of the UMI of the 5′ adapter is 5′-(rN)₁₀rArCrArCrNrNrNrNrN-3′. In some embodiments, the sequence of the UMI of the 5′ adapter is 5′-(rN)₁₁rArCrArCrNrNrNrNrN-3′. In some embodiments, the sequence of the UMI of the 5′ adapter is 5′-(rN)₁₂rArCrArCrNrNrNrNrN-3′. In some embodiments, the sequence of the UMI of the 5′ adapter is 5′-(rN)₁₃rArCrArCrNrNrNrNrN-3′. In some embodiments, the sequence of the UMI of the 5′ adapter is 5′-(rN)₁₄rArCrArCrNrNrNrNrN-3′. In some embodiments, the sequence of the UMI of the 5′ adapter is 5′-(rN)₁₅rArCrArCrNrNrNrNrN-3′. In some embodiments, the UMI of the 5′ adapter is 5′-(rN)₁₆rArCrArCrNrNrNrNrN-3′. In particular embodiments, the sequence of the UMI of the 5′ adapter is 5′-(rN)₁₃rRrYrRrYrN-3′. In particular embodiments, the sequence of the UMI of the 5′ adapter is 5′-(rN)₁₃rArCrArCrN-3′. In some embodiments, the sequence of the 5′ adapter is 5′-rGrUrUrCrArGrArGrUrUrCrUrArCrArGrUrCrCrGrArCrGrArUrCrN₁₃rArCrArCrN-3′ (SEQ ID NO: 3).

The 3′ adapter comprises at least one random (N) nucleotide at its 5′ end. The random nucleotide at the point of ligation minimizes adapter ligation bias. In some embodiments, the 3′ adapter comprises two, three, four, or five random nucleotides at its 5′ end. In specific embodiments, the 3′ adapter comprises four random nucleotides at its 5′ end. The 3′ adapter further comprises a unique sequence at its 3′ end, wherein the unique sequence is a complement sequence of a portion or all of a reverse primer used during the amplification step. The 3′ adapter can range in length from about 18-40 nucleotides, or from about 20-30 nucleotides. In some embodiments, the 3′ adapter is DNA. In some embodiments the 3′ adapter is pre-adenylated at its 5′ end and dideoxy-terminated at its 3′ end. In some embodiments, the 3′ adapter is 5′-NNNNTGGAATTCTCGGGTGCCAAGGddC-3′; SEQ ID NO: 1). In some embodiments, the 3′ adapter comprises 5′-rAppNNNNTGGAATTCTCGGGTGCCAAGGddC-3′ (SEQ ID NO: 2).

In some embodiments, the oligonucleotide is RNA and the 3′ adapter is ligated to the oligonucleotide before the 5′ adapter. In some embodiments, the oligonucleotide can be phosphorylated at the 5′ end prior to ligating the 3′ adapter. The phosphorylation reaction can be catalyzed by a polynucleotide kinase under suitable reaction conditions. Suitable polynucleotide kinases include T4 polynucleotide kinase and T7 polynucleotide kinase. The polynucleotide kinase can be wild-type, recombinant, or engineered. In some embodiments, the polynucleotide kinase can be T4 polynucleotide kinase. In some embodiments, the T4 polynucleotide kinase can be 3′ phosphatase minus. The phosphorylation reaction also requires ATP. In some embodiments, the phosphorylated oligonucleotide can be purified by routine means (e.g., spin column, chromatography method, etc.).

The ligation reaction can be catalyzed by a suitable ligase enzyme under suitable reaction conditions. The ligase can be wild-type, recombinant, engineered, or thermostable. Suitable ligase enzymes include T4 RNA ligase 1, T4 RNA ligase 2, truncated T4 RNA ligase 2, truncated T4 RNA ligase 2 KQ, 5′ App DNA/RNA ligase, RtcB ligase, T4 DNA ligase, T3 DNA ligase, T7 DNA ligase, and E. coli DNA ligase. In some embodiments, ligation of the 3′ adapter can be catalyzed by T4 RNA ligase 2. In some embodiments, ligation of the 5′ adapter can be catalyzed by T4 RNA ligase 1.

In some embodiments, the adapter-ligated product can be purified by routine means prior to the next step. In some embodiments, the adapter-ligated product can be purified by chromatography, magnetic bead purification, spin column purification, and the like.

### Amplifying, Sequencing, and Analyzing

The next step of the method comprises amplifying the plurality of adapter-ligated products using a forward primer and a reverse primer to generate a library. The methods may also comprise adding sequencing adapters, and optionally barcodes, during the amplification process to create the library, diluting the library to generate a certain number of input molecules, and re-amplifying the input molecules to generate sufficient material for sequencing.

Amplification reactions, such as PCR, are well-known in the art. In general, the amplification method comprises forming a reaction mixture comprising at least the adapter-ligated product, forward and reverse primers, dNTPs, and a (thermostable) DNA polymerase and then cycling the reaction mixture through steps of denaturation, annealing, and extension.

In embodiments in which the oligonucleotide is RNA, the plurality of adapter-ligated products is reverse transcribed to form a plurality of first strand cDNAs before amplification. The reverse transcription reaction is catalyzed by a reverse transcriptase (RT) enzyme in the presence of a reverse primer and dNTPs. The RT reaction can be performed in the presence of an RNase inhibitor. The reverse transcriptase enzyme can be wild-type, recombinant, or engineered (e.g., to improve thermostability, reduce RNase H activity, etc.) The reverse transcriptase (RT) can be MMLV RT or AMV RT, or a derivative, modified version, or variant thereof. The temperature of the RT reaction can range from about 25-60° C., or at about 42°. In some embodiments, The RT enzyme can be heat denatured once the reaction is completed.

In some embodiments, the plurality of first strand cDNAs can be purified by routine means (e.g., magnetic bead purification, spin column purification) prior to the next step.

In some embodiments, the amplification reaction synthesizes a plurality of second strand cDNAs from the plurality of first strand cDNAs. In some embodiments, the plurality of first strand cDNAs and second strand cDNAs are amplified in a first amplifying reaction to generate a preliminary library of amplified adapter-ligated products using a forward primer and a reverse barcode primer. In some embodiments, the synthesis of the second strand cDNAs occurs in the first amplifying reaction.

In some embodiments, the forward primer incorporates a 5′ sequencing adapter sequence, and optionally a bar code, to the 5′ end of the amplified adapter-ligated products, and the reverse barcode primer incorporates a 3′ sequencing adapter sequence, and optionally a barcode, to the 3′ end of the amplified adapter-ligated products. In some embodiments, N number of different samples of oligonucleotides can be characterized in N number of different reactions, wherein each reaction incorporates a different barcode into the amplified adapter-ligated products. The number of different barcodes can be equal to the N number of different reactions. The addition of a single (3′ or 5′) or dual barcode pair (3′ and 5′) to the adapter-ligated product can enable the association of the particular oligonucleotide species and reaction from which the barcoded nucleic acid sequence was derived especially when the N number of different reactions are pooled and sequenced together. For example, an oligonucleotide sequence identified by the barcode can enable the identification of the reaction and thus the sample of oligonucleotides associated with the barcode.

In some embodiments, the barcode sequences can comprise from about 4 to about 10 or more nucleotides. In some cases, the length of a barcode sequence can be about 4, 5, 6, 7, 8, 9, 10 nucleotides, or longer. In some cases, the length of a barcode sequence can be at least about 4, 5, 6, 7, 8, 9, 10 nucleotides, or longer. In some cases, the length of a barcode sequence can be at most about 4, 5, 6, 7, 8, 9, 10 nucleotides, or shorter.

In some embodiments, the barcodes can be 3′ single index barcodes. In some embodiments, the barcodes can be 5′/3′ dual index barcodes. In some embodiments, the barcodes are short sequences comprising 4, 5, 6, 7, or 8 nucleotides. In specific embodiments, the barcodes utilize a single indexed adapter containing a 6-nucleotide unique sequence. Commercially available kits comprising reverse barcode primers that comprise the barcodes and the 3′ sequencing adapter sequence can be used in the methods described herein. Suitable reverse barcode primers for use in the methods described herein include NEXTFLEX® barcodes sets A and B (PerkinElmer).

In some embodiments, the preliminary library is diluted to about 10,000-200,000 molecules. In some embodiments, the preliminary library is diluted to about 10,000, about 20,000, to about 30,000, to about 40,000, or about 50,000 molecules. In some embodiments, the preliminary library is diluted to about 10,000 molecules. In some embodiments, a second amplification reaction is performed for the diluted library to generate a library of amplified adapter-ligated products using the forward primer and the reverse barcode primer used to generate sequencing library for the reaction.

The temperature and duration of the steps can and will vary. In general, the denaturation temperature can range from about 94-98° C., the annealing temperature depends upon the melting temperature (Tm) of the primers and can range from about 48-72° C., and the extension temperature can range from about 68-72° C. The thermostable DNA polymerase can be recombinant and/or engineered for improved fidelity, stability, performance, etc. The thermostable DNA polymerase can be a Taq, Pfu, Pfx, Bst, Tfi, Tth DNA polymerase or derivative, modified version, or variant thereof. In some embodiments, the thermostable DNA polymerase can be a high-fidelity polymerase (e.g., Phusion polymerase; NEB).

In some embodiments, the library can be purified by routine means (e.g., magnetic bead purification, spin column purification) prior to the next step.

The next step of the method comprises sequencing the library to generate sequencing fragments of forward and reverse reads of the adapter-ligated products. The amplified adapter-ligated product comprises the UMI, the oligonucleotide, and the at least one random nucleotide from the 3′ adapter. In some embodiments, libraries from two or more different reactions can be pooled before sequencing the libraries, wherein each library has a different single barcode or dual barcode pair incorporated into the amplified adapter-ligated products. In some embodiments, the libraries are pooled in equimolar ratios to generate a sequencing pool. In general, the sequencing method is a high throughput, massively parallel, deep sequencing method (i.e., next generation sequencing). In some embodiments, the sequencing method comprises a next generation sequencing platform. In some embodiments, the sequencing platform can be MiSeq (from Illumina), Roche 454, GS FLX Titanium, Illumina HiSeq, Illumina NextSeq, Illumina Genome analyzer IIX, Life Technologies SOLiD4, Life Technologies Ion Proton, Complete Genomics, Helicos Biosciences, Heliscope, Pacific Biosciences SMRT, or Ion Torrent PGM. As such, preparation and sequencing of the library is performed according to the manufacturer's instructions.

The sequencing fragments are analyzed and processed to determine the nucleotide sequences of the plurality of the oligonucleotides and the relative abundance of the nucleotide sequences thereby characterizing the oligonucleotides in the sample. In some embodiments, sequencing fragments having the same UMI are binned and counted. In some embodiments, a fixed sequence, e.g., RYRY or ACAC, can be used to filter out sequences that have a faulty UMI. In some embodiments, sequences with the same UMI sequences are deduplicated, the 5′ adapter sequences, which include the UMI sequences, and the 3′ adapter sequences are trimmed from the sequence fragments thereby generating corresponding nucleotide sequences of the plurality of oligonucleotides, and/or the nucleotide sequences of the plurality of oligonucleotides are compared to a reference oligonucleotide sequence to identify full-length oligonucleotides with no sequence variation relative to the reference oligonucleotide. In some embodiments, at least about 5-1000, at least about 5-500, at least about 5-100, at least about 5-50, at least about 5-20, at least about 10-1000, at least about 10-500, at least about 10-100, at least about 10-50, at least about 10-20, at least about 15-1000, at least about 15-500, at least about 15-100, at least about 15-50, at least about 15-20, at least about 20-1000, at least about 20-500, at least about 20-100, at least about 20-50, at least about 10-30, at least about 15-25, at least about 50-1000, at least about 50-500, at least about 100-1000, at least about 100-500, at least about 200-750, or at least about 100-200 reads of the UMI are performed to generate a consensus sequence. In some embodiments, at least about 5, at least about 10, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 35, at least about 40, at least about 50, at least about 75, at least about 100, at least about 125, at least about 150, at least about 200, at least about 300, at least about 400, at least about 500, at least about 600, at least about 700, at least about 800, at least about 900, or at least about 1000 reads of the UMI are performed to generate a consensus sequence.

The raw sequencing data of the sequencing fragments can be analyzed using a variety of commercial, freeware, and proprietary tools. The analyzing comprises trimming of adapter sequences and trimming of degenerate bases on the 3′ end of the library, merging of forward and reverse reads into a consensus sequence, binning sequencing fragments having the same UMI sequence at the 5′ end and generating a consensus sequence-and performing alignment with starting oligonucleotide or reference sequence. From this analysis, the relative proportion of full-length accurate sequences can be determined. Additionally, sequence variants (e.g., 5′ truncated sequences, 3′ truncated sequences, and/or sequences comprising or more substitution, insertion, and/or deletion) can be identified and the relative abundance of each can be determined. The method provides a thorough analysis of the purity and/or quality of the oligonucleotide.

For example, a Linux/Python-based pipeline can be used for processing raw NGS data; a Trimmomatic tool (Bolger, A. M., Lohse, M., & Usadel, B. (2014), Trimmomatic: A flexible trimmer for Illumina Sequence Data. Bioinformatics, btu170), BBduk, BBmap, or other BBtools can be used to trim adaptors and degenerate bases on the 3′ end of the library; PEAR (Paired-End read merger), COPE (connecting overlapping paired end reads), BBMerge, FLASH (fast length adjustment of short reads), PANDAseq, BBmap, Usearch or other BBtools can be used to combine forward and reverse reads into a consensus sequence; AmpUMI (Clement et al., Bioinformatics, 2018, 34, i202-i210), UMI tools, BBmap or other BBtools can be used to deduplicate UMIs; and Needleman-Wunsch algorithm (e.g., the Emboss Needleall alignment tool), BBMap, or other BBtools can be used to align the sequences with a reference sequence.

### II. METHODS FOR CHARACTERIZING SMALL RNAS

Also provided herein are methods for characterizing small RNAs, e.g., small RNA purity. The methods comprise (a) providing a sample comprising a plurality of small RNAs and (b) ligating a plurality of 3′ adapters to the plurality of small RNAs to generate a plurality of 3′-ligated products. The 3′ adapter comprises at least one random nucleotide at its 5′ end and a unique sequence at its 3′ end. The unique sequence comprises a complement sequence of a portion or all of a reverse primer. The methods further comprise (c) ligating a plurality of 5′ adapters to the plurality of 3′-ligated products to generate a plurality of 5′- and 3′-ligated products, the 5′ adapter comprising a unique molecular identifier (UMI) and a unique sequence located 5′ to the UMI. The UMI comprises (5′-3′) about 10-16 random nucleotides, about 3-6 semi-random nucleotides, and one random nucleotide. For example, the sequence of the UMI can be 5′-(N)₁₀-₁₆RYRY(N)₁₋₅-3′, 5′-(N)₁₀₋₁₆ACAC(N)₁₋₅-3′, wherein N is A, C, G, or T/U, R is A or G, and Y is C or T/U, 5′-(rN)₁₀₋₁₆rRrYrRrY(rN)₁₋₅-3′, or 5′-(rN)₁₀₋₁₆rArCrArC(rN)₁₋₅-3′, wherein rN is rA, rC, rG, or rU, rR is rA or rG, and rY is rC or rU. The unique sequence corresponds to a portion or all of a forward primer. The methods further comprise (d) reverse transcribing the plurality of 5′- and 3′-ligated products with the reverse primer to generate a plurality of first strand cDNA and (e) synthesizing a plurality of second strand cDNAs from the plurality of first strand cDNAs. The methods further comprise (f) amplifying the plurality of first strand and second strand cDNAs in a first amplifying reaction to generate a preliminary library of amplified 5′- and 3′-ligated products using a forward primer and a reverse barcode primer. In some embodiments, step (e) occurs concurrently with step (f). The forward primer incorporates a 5′ sequencing adapter sequence, and optionally a barcode, to the 5′ end of the amplified 5′- and 3′-ligated products. The reverse barcode primer incorporates a 3′ sequencing adapter sequence, and optionally a barcode, to the 3′ end of the amplified 5′- and 3′-ligated products. The methods further comprise (g) diluting the preliminary library to about 10,000-50,000 molecules and performing a second amplification reaction to generate a library of amplified 5′- and 3′-ligated products using the forward primer and the reverse barcode primer. The methods further comprise (h) sequencing the library to generate sequencing fragments of forward and reverse reads of the amplified 5′- and 3′-ligated products, wherein the amplified 5′- and 3′-ligated product comprises the UMI, the small RNA, and the at least one random nucleotide from the 3′ adapter; and (i) analyzing and processing the sequencing fragments to determine the nucleotide sequences of the plurality of the small RNAs and the relative abundance of the nucleotide sequences thereby characterizing the small RNAs in the sample.

In some embodiments, the sequencing fragments are counted and binned based on the UMI. In some embodiments, sequences with the same UMI sequences are deduplicated, the 5′ adapter sequences, which include the UMI sequences, and the 3′ adapter sequences are trimmed from the sequence fragments thereby generating corresponding nucleotide sequences of the plurality of small RNAs, and the nucleotide sequences of the plurality of small RNAs are compared to a reference small RNA sequence to identify full-length small RNAs with no sequence variation relative to the reference small RNA. In some embodiments, analyzing the sequencing fragments further comprises identifying and quantifying sequence variants. In some embodiments, the sequence variants comprise 5′ truncated sequences, 3′ truncated sequences, sequences comprising a substitution, insertion, and/or deletion of at least one nucleotide, or a combination thereof.

In some embodiments, the 5′ adapter comprises 5′-(N)₁₀₋₁₆RYRY(N)₁₋₅-3′ and the sequencing fragments are filtered for the presence of the nucleotide sequence RYRY before trimming the 5′ adapter sequences, which include the UMI sequences, and the 3′ adapter sequences from the sequencing fragments. In some embodiments, the 5′ adapter comprises 5′-(N)₁₀₋₁₆ACAC(N)₁₋₅-3 and the sequencing fragments are filtered for the presence of the nucleotide sequence ACAC before trimming the 5′ adapter sequences, which include the UMI sequences, and the 3′ adapter sequences from the sequencing fragments.

In embodiments in which the small RNA is synthetic, the characterizing can comprise determining the purity of the small RNA, e.g., the relative abundance of full-length accurate sequences and/or the relative abundance of sequence variants. In embodiments in which the small RNA is naturally occurring, the characterizing can comprise profiling the small RNA population with regard to sequence diversity and/or abundance.

### Small RNA

The methods of the disclosure can be used to characterize, e.g., determine the purity of, a variety of small RNAs. For example, the small RNA can be a synthetic RNA, or the small RNA can be a naturally occurring small RNA (e.g., cellular RNAs from biological sources).

In some embodiments, the small RNA is synthetic. Suitable synthetic RNA includes gRNA (e.g., sgRNA or crRNA), miRNA, siRNA, shRNA, antisense RNA, RNA adapter, RNA primer, or RNA probe. Synthetic RNA can generally be prepared using phosphoramidite chemistry, in vitro transcription, or a combination thereof. In other embodiments, the small RNA is a naturally occurring small RNA. Examples of suitable naturally occurring small RNAs include miRNA, siRNA, or piRNA.

The small RNA can comprise standard nucleobases, such adenine (A), guanine (G), cytosine (C), and uracil (U). In some embodiments, the oligonucleotide can comprise modified natural nucleobases, such as 5-methylcytosine (5meC), 5-(hydroxymethyl)cytosine (5hmC), 5-formylcytosine (5fC), 5-carboxycytosine (5caC), 5-(hydroxymethyl)uracil (5hmU), 5-formyluracil (5fU), dihydrouracil, pseudouracil, N6-methyladenine (5mA), xanthine, hypoxanthine, 7-methylguanine, and so forth. In some embodiments, the small RNA can comprise one or more substituted sugar moieties, e.g., one of the following at the 2′ position: OH, SH, SCH₃, F, OCN, OCH³, OCH₃O(CH₂)nCH₃, O(CH₂)nNH₂, or O(CH₂)nCH₃, where n is from 1 to about 10, alkyl or O-, S-, or N-alkyl, wherein alkyl is C1 to C10 alkyl. Similar modifications can be made at the 3′ position of the sugar on the 3′ terminal nucleotide of any small RNA and/or the 5′ position of the 5′ terminal nucleotide of any small RNA. The small RNA can comprise standard phosphodiester linkages and/or modified linkages such as phosphorothioates, phosphotriesters, morpholinos, methyl phosphonates, locked nucleic acids (LNA), peptide nucleic acids (PNA), short chain alkyl or cycloalkyl intersugar linkages, or short chain heteroatomic or heterocyclic intersugar linkages.

The small RNA can range in length from about 15-200 nucleotides. In some embodiments, the small RNA can range in length from 15-30 nt, from about 20-27 nt, from about 26-31 nt, from about 30-50 nt, from about 40-60 nt, from about 50-100 nt, from about 90-110 nt, from about 80-120 nt, from about 100-150 nt, from about 120-180 nt, or from about 150-200 nt.

### Phosphorylating Small RNA

In some embodiments, the small RNA can be phosphorylated at the 5′ end prior to ligating the 3′ adapter. The phosphorylation reaction can be catalyzed by a polynucleotide kinase under suitable reaction conditions. The polynucleotide kinase can be wild-type, recombinant, or engineered. Suitable kinases include T4 polynucleotide kinase and T7 polynucleotide kinase. In some embodiments, the polynucleotide kinase can be T4 polynucleotide kinase. In some embodiments, the T4 polynucleotide kinase can be 3′ phosphatase minus. The phosphorylation reaction also requires ATP. In some embodiments, the phosphorylated small RNA can be purified by routine means (e.g., spin column, chromatography method, etc.).

### Ligating 3′ Adapter

The disclosed methods involve providing a sample comprising a plurality of small RNAs and ligating a plurality of 3′ adapters to the plurality of small RNAs to generate a plurality of 3′-ligated products, wherein the 3′ adapter comprises at least one random nucleotide at its 5′ end. The 3′ adapter is ligated to the 3′ end of the small RNA. The random nucleotide at the 5′ end of the 3′ adapter (or point of ligation) minimizes adapter ligation bias. In general, the 3′ adapter comprises deoxyribonucleotides (DNA). In some embodiments, the 3′ adapter comprises two, three, four, or five random (N) nucleotides at its 5′ end. In specific embodiments, the 3′ adapter comprises four random nucleotides at its 5′ end. The 3′ adapter further comprise a unique sequence at its 3′ end, wherein the unique sequence is a complement of a portion or all of a reverse primer used during the reverse transcription step. In some embodiments, the 3′ adapter is pre-adenylated at the 5′ end. In some embodiments, the 3′ adapter comprises a dideoxy nucleotide at the 3′ end. In certain embodiments, the dideoxy nucleotide can be ddC. The overall length of the 3′ adapter can range from about 18-40 nucleotides, or from about 20-30 nucleotides. In some embodiments, the 3′ adapter is 5′-NNNNTGGAATTCTCGGGTGCCAAGGddC-3′ (SEQ ID NO: 1). In some embodiments, the 3′ adapter comprises 5′-rAppNNNNTGGAATTCTCGGGTGCCAAGGddC-3′ (SEQ ID NO: 2). In some embodiments, the 3′ adapter is ligated to the small RNA first before the 5′ adapter to ensure ligation to the 3′ of the small RNA.

Ligation of the 3′ adapter is catalyzed by a suitable RNA ligase, such as, for example, T4 RNA ligase 1, T4 RNA ligase 2, truncated T4 RNA ligase 2, truncated T4 RNA ligase 2 KQ, 5′ App DNA/RNA ligase, or. RtcB ligase. The ligase can be wild-type, recombinant, engineered, or thermostable. In general, ligation of the 3′ adapter is conducted in the presence of T4 RNA ligase 2 under suitable reaction conditions. In some embodiments, the T4 RNA ligase 2 can be a truncated T4 RNA ligase 2. In some embodiments, the T4 RNA ligase 2 can be a truncated T4 RNA ligase 2 KQ. The temperature of the ligation reaction can range from about 4-37° C. In specific embodiments, the temperature of the reaction can be about 25° C. The ligase can be heat denatured once the reaction is completed.

In some embodiments, the 3′-ligated product can be purified by routine means (e.g., magnetic bead purification, spin column purification, chromatography methods, etc.) prior to the next step.

### Ligating 5′ Adapter

The next step of the method comprises ligating a plurality of 5′ adapters to the plurality of 3′-ligated products to generate a plurality of 5′- and 3′-ligated products. The 5′ adapter is ligated to the 5′ end of the 3′-ligated product. The 5′ adapter comprises a unique molecular identifier (UMI) comprising (5′-3′) about 10-16 random nucleotides, about 3-6 semi-random nucleotides, and at least one random nucleotide. The random nucleotide at the 3′ end of the 5′ adapter (or point of ligation) minimizes ligation bias. In general, the 5′ adapter comprised ribonucleotides (RNA). In some embodiments, the UMI comprises (5′-3′) about 12-14 random nucleotides, about 3-5 semi-random nucleotides, and at least one random nucleotide.

In some embodiments, the sequence of the UMI is 5′-(N)₁₀RYRYN-3′, wherein N is A, C, G, or T/U, R is A or G, and Y is C or T/U. In some embodiments, the sequence of the UMI is 5′-(N)₁₁RYRYN-3′. In some embodiments, the sequence of the UMI is 5′-(N)₁₂RYRYN-3′. In some embodiments, the sequence of the UMI is 5′-(N)₁₃RYRYN-3′. In some embodiments, the sequence of the UMI is 5′-(N)₁₄RYRYN-3′. In some embodiments, the sequence of the UMI is 5′-(N)₁₅RYRYN-3′. In some embodiments, the sequence of the UMI is 5′-(N)₁₆RYRYN-3′. In some embodiments, the sequence of the UMI is 5′-(N)₁₀RYRYNN-3′. In some embodiments, the sequence of the UMI is 5′-(N)₁₁RYRYNN-3′. In some embodiments, the sequence of the UMI is 5′-(N)₁₂RYRYNN-3′. In some embodiments, the sequence of the UMI is 5′-(N)₁₄RYRYNN-3′. In some embodiments, the sequence of the UMI is 5′-(N)₁₄RYRYNN-3′. In some embodiments, the sequence of the UMI is 5′-(N)₁₅RYRYNN-3′. In some embodiments, the sequence of the UMI is 5′-(N)₁₆RYRYNN-3′. In some embodiments, the sequence of the UMI is 5′-(N)₁₀RYRYNNN-3′. In some embodiments, the sequence of the UMI is 5′-(N)₁₁RYRYNNN-3′. In some embodiments, the sequence of the UMI is 5′-(N)₁₂RYRYNNN-3′. In some embodiments, the sequence of the UMI is 5′-(N)₁₃RYRYNNN-3′. In some embodiments, the sequence of the UMI is 5′-(N)₁₄RYRYNNN-3′. In some embodiments, the sequence of the UMI is 5′-(N)₁₅RYRYNNN-3′. In some embodiments, the sequence of the UMI is 5′-(N)₁₆RYRYNNN-3′. In some embodiments, the sequence of the UMI is 5′-(N)₁₀RYRYNNNN-3′. In some embodiments, the sequence of the UMI is 5′-(N)₁₁RYRYNNNN-3′. In some embodiments, the sequence of the UMI is 5′-(N)₁₂RYRYNNNN-3′. In some embodiments, the sequence of the UMI is 5′-(N)₁₃RYRYNNNN-3′. In some embodiments, the sequence of the UMI is 5′-(N)₁₄RYRYNNNN-3′. In some embodiments, the sequence of the UMI is 5′-(N)₁₅RYRYNNNN-3′. In some embodiments, the sequence of the UMI is 5′-(N)₁₆RYRYNNNN-3′. In some embodiments, the sequence of the UMI is 5′-(N)₁₀RYRYNNNNN-3′. In some embodiments, the sequence of the UMI is 5′-(N)₁₁RYRYNNNNN-3′. In some embodiments, the sequence of the UMI is 5′-(N)₁₂RYRYNNNNN-3′. In some embodiments, the sequence of the UMI is 5′-(N)₁₃RYRYNNNNN-3′. In some embodiments, the sequence of the UMI is 5′-(N)₁₄RYRYNNNNN-3′. In some embodiments, the sequence of the UMI is 5′-(N)₁₅RYRYNNNNN-3′. In some embodiments, the sequence of the UMI is 5′-(N)₁₆RYRYNNNNN-3′. In some embodiments, the sequence of the UMI is 5′-(N)₁₀ACACN-3′. In some embodiments, the sequence of the UMI is 5′-(N)₁₁ACACN-3′. In some embodiments, the sequence of the UMI is 5′-(N)₁₂ACACN-3′. In some embodiments, the sequence of the UMI is 5′-(N)₁₃ACACN-3′. In some embodiments, the sequence of the UMI is 5′-(N)₁₄ACACN-3′. In some embodiments, the sequence of the UMI is 5′-(N)₁₅ACACN-3′. In some embodiments, the sequence of the UMI is 5′-(N)₁₆ACACN-3′. In some embodiments, the sequence of the UMI is 5′-(N)₁₀ACACNN-3′. In some embodiments, the sequence of the UMI is 5′-(N)₁₁ACACNN-3′. In some embodiments, the sequence of the UMI is 5′-(N)₁₂ACACNN-3′. In some embodiments, the sequence of the UMI is 5′-(N)₁₃ACACNN-3′. In some embodiments, the sequence of the UMI is 5′-(N)₁₄ACACNN-3′. In some embodiments, the sequence of the UMI is 5′-(N)₁₅ACACNN-3′. In some embodiments, the sequence of the UMI is 5′-(N)₁₆ACACNN-3′. In some embodiments, the sequence of the UMI is 5′-(N)₁₀ACACNNN-3′. In some embodiments, the sequence of the UMI is 5′-(N)₁₁ACACNNN-3′. In some embodiments, the sequence of the UMI is 5′-(N)₁₂ACACNNN-3′. In some embodiments, the sequence of the UMI is 5′-(N)₁₃ACACNNN-3′. In some embodiments, the sequence of the UMI is 5′-(N)₁₄ACACNNN-3′. In some embodiments, the sequence of the UMI is 5′-(N)₁₅ACACNNN-3′. In some embodiments, the sequence of the UMI is 5′-(N)₁₆ACACNNN-3′. In some embodiments, the sequence of the UMI is 5′-(N)₁₀ACACNNNN-3′. In some embodiments, the sequence of the UMI is 5′-(N)₁₁ACACNNNN-3′. In some embodiments, the sequence of the UMI is 5′-(N)₁₂ACACNNNN-3′. In some embodiments, the sequence of the UMI is 5′-(N)₁₃ACACNNNN-3′. In some embodiments, the sequence of the UMI is 5′-(N)₁₄ACACNNNN-3′. In some embodiments, the sequence of the UMI is 5′-(N)₁₅ACACNNNN-3′. In some embodiments, the sequence of the UMI is 5′-(N)₁₆ACACNNNN-3′. In some embodiments, the sequence of the UMI is 5′-(N)₁₀ACACNNNNN-3′. In some embodiments, the sequence of the UMI is 5′-(N)₁₁ACACNNNNN-3′. In some embodiments, the sequence of the UMI is 5′-(N)₁₂ACACNNNNN-3′. In some embodiments, the sequence of the UMI is 5′-(N)₁₃ACACNNNNN-3′. In some embodiments, the sequence of the UMI is 5′-(N)₁₄ACACNNNNN-3′. In some embodiments, the sequence of the UMI is 5′-(N)₁₅ACACNNNNN-3′. In some embodiments, the sequence of the UMI is 5′-(N)₁₆ACACNNNNN-3′. In some particular embodiments, the sequence of the UMI is 5′-(N)₁₃RYRYN-3′. In further specific embodiments, the sequence of the UMI is 5′-(N)₁₃ACACN-3′. In some embodiments, the sequence of the UMI is 5′-GTTCAGAGTTCTACAGTCCGACGATCNNNNNNNNNNNNNACAC-3′ (SEQ ID NO: 4). The 5′ adapter further comprises a unique sequence located 5′ to the UMI, wherein the unique sequence corresponds to a portion or all of a forward primer used during the amplification step. The overall length of the 5′ adapter can range from about 30-60 nucleotides, from about 35-50 nucleotides, or from about 40-45 nucleotides.

In some embodiments, the 5′ adapter is RNA and the sequence of the UMI comprises 5′-(rN)₁₀rRrYrRrYrN-3′, wherein rN is rA, rC, rG, or rU, rR is rA or rG, and rY is rC or rU. In some embodiments, the sequence of the UMI of the 5′ adapter is 5′-(rN)₁₁rRrYrRrYrN-3′. In some embodiments, the sequence of the UMI of the 5′ adapter is 5′-(rN)₁₂rRrYrRrYrN-3′. In some embodiments, the sequence of the UMI of the 5′ adapter is 5′-(rN)₁₃rRrYrRrYrN-3′. In some embodiments, the sequence of the UMI of the 5′ adapter is 5′-(rN)₁₄oRrYrRrYrN-3′. In some embodiments, the sequence of the UMI of the 5′ adapter is 5′-(rN)₁₅rRrYrRrYrN-3′. In some embodiments, the sequence of the UMI of the 5′ adapter is 5′-(rN)₁₆rRrYrRrYrN-3′. In some embodiments, the sequence of the UMI of the 5′ adapter is 5′-(rN)₁₀rRrYrRrYrNrN-3′. In some embodiments, the sequence of the UMI of the 5′ adapter is 5′-(rN)₁₁rRrYrRrYrNrN-3′. In some embodiments, the sequence of the UMI of the 5′ adapter is 5′-(rN)₁₂rRrYrRrYrNrN-3′. In some embodiments, the sequence of the UMI of the 5′ adapter is 5′-(rN)₁₃rRrYrRrYrNrN-3′. In some embodiments, the sequence of the UMI of the 5′ adapter is 5′-(rN)₁₄rRrYrRrYrNrN-3′. In some embodiments, the sequence of the UMI of the 5′ adapter is 5′-(rN)₁₅rRrYrRrYrNrN-3′. In some embodiments, the sequence of the UMI of the 5′ adapter is 5′-(rN)₁₆rRrYrRrYrNrN-3′. In some embodiments, the sequence of the UMI of the 5′ adapter is 5′-(rN)₁₀rRrYrRrYrNrNrN-3′. In some embodiments, the sequence of the UMI of the 5′ adapter is 5′-(rN)₁₁rRrYrRrYrNrNrN-3′. In some embodiments, the sequence of the UMI of the 5′ adapter is 5′-(rN)₁₂rRrYrRrYrNrNrN-3′. In some embodiments, the sequence of the UMI of the 5′ adapter is 5′-(rN)₁₃rRrYrRrYrNrNrN-3′. In some embodiments, the sequence of the UMI of the 5′ adapter is 5′-(rN)₁₄rRrYrRrYrNrNrN-3′. In some embodiments, the sequence of the UMI of the 5′ adapter is 5′-(rN)₁₅rRrYrRrYrNrNrN-3′. In some embodiments, the sequence of the UMI of the 5′ adapter is 5′-(rN)₁₆rRrYrRrYrNrNrN-3′. In some embodiments, the sequence of the UMI of the 5′ adapter is 5′-(rN)₁₀rRrYrRrYrNrNrNrN-3′. In some embodiments, the sequence of the UMI of the 5′ adapter is 5′-(rN)₁₁rRrYrRrYrNrNrNrN-3′. In some embodiments, the sequence of the UMI of the 5′ adapter is 5′-(rN)₁₂rRrYrRrYrNrNrNrN-3′. In some embodiments, the sequence of the UMI of the 5′ adapter is 5′-(rN)₁₃rRrYrRrYrNrNrNrN-3′. In some embodiments, the sequence of the UMI of the 5′ adapter is 5′-(rN)₁₄rRrYrRrYrNrNrNrN-3′. In some embodiments, the sequence of the UMI of the 5′ adapter is 5′-(rN)₁₅rRrYrRrYrNrNrNrN-3′. In some embodiments, the sequence of the UMI of the 5′ adapter is 5′-(rN)₁₆rRrYrRrYrNrNrNrN-3′. In some embodiments, the sequence of the UMI of the 5′ adapter is 5′-(rN)₁₀rRrYrRrYrNrNrNrNrN-3′. In some embodiments, the sequence of the UMI of the 5′ adapter is 5′-(rN)₁₁rRrYrRrYrNrNrNrNrN-3′. In some embodiments, the sequence of the UMI of the 5′ adapter is 5′-(rN)₁₂rRrYrRrYrNrNrNrNrN-3′. In some embodiments, the sequence of the UMI of the 5′ adapter is 5′-(rN)₁₃rRrYrRrYrNrNrNrNrN-3′. In some embodiments, the sequence of the UMI of the 5′ adapter is 5′-(rN)₁₄rRrYrRrYrNrNrNrNrN-3′. In some embodiments, the sequence of the UMI of the 5′ adapter is 5′-(rN)₁₅rRrYrRrYrNrNrNrNrN-3′. In some embodiments, the sequence of the UMI of the 5′ adapter is 5′-(rN)₁₆rRrYrRrYrNrNrNrNrN-3′. In some embodiments, the sequence of the UMI of the 5′ adapter is 5′-(rN)₁₀rArCrArCrN-3′. In some embodiments, the sequence of the UMI of the 5′ adapter is 5′-(rN)₁₁rArCrArCrN-3′. In some embodiments, the sequence of the UMI of the 5′ adapter is 5′-(rN)₁₂rArCrArCrN-3′. In some embodiments, the sequence of the UMI of the 5′ adapter is 5′-(rN)₁₃rArCrArCrN-3′. In some embodiments, the sequence of the UMI of the 5′ adapter is 5′-(rN)₁₄rArCrArCrN-3′. In some embodiments, the sequence of the UMI of the 5′ adapter is 5′-(rN)₁₅rArCrArCrN-3′. In some embodiments, the UMI of the 5′ adapter is 5′-(rN)₁₆rArCrArCrN-3′. In some embodiments, the sequence of the UMI of the 5′ adapter is 5′-(rN)₁₀rArCrArCrNrN-3′. In some embodiments, the sequence of the UMI of the 5′ adapter is 5′-(rN)₁₁rArCrArCrNrN-3′. In some embodiments, the sequence of the UMI of the 5′ adapter is 5′-(rN)₁₂rArCrArCrNrN-3′. In some embodiments, the sequence of the UMI of the 5′ adapter is 5′-(rN)₁₃rArCrArCrNrN-3′. In some embodiments, the sequence of the UMI of the 5′ adapter is 5′-(rN)₁₄rArCrArCrNrN-3′. In some embodiments, the sequence of the UMI of the 5′ adapter is 5′-(rN)₁₅rArCrArCrNrN-3′. In some embodiments, the UMI of the 5′ adapter is 5′-(rN)₁₆rArCrArCrNrN-3′. In some embodiments, the sequence of the UMI of the 5′ adapter is 5′-(rN)₁₀rArCrArCrNrNrN-3′. In some embodiments, the sequence of the UMI of the 5′ adapter is 5′-(rN)₁₁rArCrArCrNrNrN-3′. In some embodiments, the sequence of the UMI of the 5′ adapter is 5′-(rN)₁₂rArCrArCrNrNrN-3′. In some embodiments, the sequence of the UMI of the 5′ adapter is 5′-(rN)₁₃rArCrArCrNrNrN-3′. In some embodiments, the sequence of the UMI of the 5′ adapter is 5′-(rN)₁₄rArCrArCrNrNrN-3′. In some embodiments, the sequence of the UMI of the 5′ adapter is 5′-(rN)₁₅rArCrArCrNrNrN-3′. In some embodiments, the UMI of the 5′ adapter is 5′-(rN)₁₆rArCrArCrNrNrN-3′. In some embodiments, the sequence of the UMI of the 5′ adapter is 5′-(rN)₁₀rArCrArCrNrNrNrN-3′. In some embodiments, the sequence of the UMI of the 5′ adapter is 5′-(rN)₁₁rArCrArCrNrNrNrN-3′. In some embodiments, the sequence of the UMI of the 5′ adapter is 5′-(rN)₁₂rArCrArCrNrNrNrN-3′. In some embodiments, the sequence of the UMI of the 5′ adapter is 5′-(rN)₁₃rArCrArCrNrNrNrN-3′. In some embodiments, the sequence of the UMI of the 5′ adapter is 5′-(rN)₁₄rArCrArCrNrNrNrN-3′. In some embodiments, the sequence of the UMI of the 5′ adapter is 5′-(rN)₁₅rArCrArCrNrNrNrN-3′. In some embodiments, the UMI of the 5′ adapter is 5′-(rN)₁₆rArCrArCrNrNrNrN-3′. In some embodiments, the sequence of the UMI of the 5′ adapter is 5′-(rN)₁₀rArCrArCrNrNrNrNrN-3′. In some embodiments, the sequence of the UMI of the 5′ adapter is 5′-(rN)₁₁rArCrArCrNrNrNrNrN-3′. In some embodiments, the sequence of the UMI of the 5′ adapter is 5′-(rN)₁₂rArCrArCrNrNrNrNrN-3′. In some embodiments, the sequence of the UMI of the 5′ adapter is 5′-(rN)₁₃rArCrArCrNrNrNrNrN-3′. In some embodiments, the sequence of the UMI of the 5′ adapter is 5′-(rN)₁₄rArCrArCrNrNrNrNrN-3′. In some embodiments, the sequence of the UMI of the 5′ adapter is 5′-(rN)₁₅rArCrArCrNrNrNrNrN-3′. In some embodiments, the UMI of the 5′ adapter is 5′-(rN)₁₆rArCrArCrNrNrNrNrN-3′. In particular embodiments, the sequence of the UMI of the 5′ adapter is 5′-(rN)₁₃rRrYrRrYrN-3′. In particular embodiments, the sequence of the UMI of the 5′ adapter is 5′-(rN)₁₃rArCrArCrN-3′. In some embodiments, the sequence of the 5′ adapter is 5′-rGrUrUrCrArGrArGrUrUrCrUrArCrArGrUrCrCrGrArCrGrArUrCrN₁₃rArCrArCrN-3′ (SEQ ID NO: 3).

Ligation of the 5′ adapter to the 3′ -ligated products is catalyzed by a suitable RNA ligase, such as, for example, T4 RNA ligase 1, T4 RNA ligase 2, truncated T4 RNA ligase 2, truncated T4 RNA ligase 2 KQ, 5′ App DNA/RNA ligase, or. RtcB ligase. The ligase can be wild-type, recombinant, engineered, or thermostable. In general, ligation of the 5′ adapter is conducted in the presence of T4 RNA ligase 1 under suitable reaction conditions. The temperature of the ligation reaction can range from about 4-37° C. In specific embodiments, the temperature of the reaction can be about 25° C. The ligase can be heat denatured once the reaction is completed.

In some embodiments, the plurality of 5′- and 3′-ligated products can be purified by routine means (e.g., magnetic bead purification, spin column purification) prior to the next step. In some embodiments, the plurality of 5′- and 3′-ligated products can be purified by chromatography, magnetic bead purification, spin column purification, and the like.

### Reverse Transcribing

The next step of the method comprises reverse transcribing the 5′- and 3′-ligated products to form first strand cDNA. The reverse transcription reaction is catalyzed by a reverse transcriptase (RT) enzyme in the presence of a reverse primer, and dNTPs. In some embodiments, the RT reaction can be performed in the presence of an RNase inhibitor. In some embodiments, the reverse transcriptase enzyme can be wild-type, recombinant, or engineered (e.g., to improve thermostability, reduce RNase H activity, etc.) In some embodiments, the reverse transcriptase (RT) can be MMLV RT or AMV RT, or a derivative, modified version, or variant thereof. In some embodiments, the temperature of the RT reaction can range from about 25-60° C., or at about 42° . In some embodiments, the RT enzyme can be heat denatured once the reaction is completed.

In some embodiments, the first strand cDNA can be purified by routine means (e.g., magnetic bead purification, spin column purification) prior to the next step.

Synthesizing Second Strand cDNA and Amplifying

The next step of the method comprises synthesizing second strand cDNA and amplifying the 5′- and 3′-ligated products to generate a preliminary library of amplified 5′ and 3′ ligated products. For this, the first strand cDNA is contacted with a forward sequencing primer, whose sequence corresponds to a portion of the 5′ adapter, dNTPs, and a DNA polymerase under suitable reaction conditions. During the next step of the method, the cDNA is contacted with forward and reverse barcode primers, dNTPs, and DNA polymerase essentially as described above.

The plurality of first strand and second strand cDNAs are amplified in a first amplifying reaction to generate a preliminary library of amplified 5′- and 3′-ligated products using a forward primer and a reverse barcode primer. The forward primer incorporates a 5′ sequencing adapter sequence, and optionally a barcode to the 5′ end of the amplified 5′- and 3′-ligated products. The reverse barcode primer incorporates a 3′ sequencing adapter sequence, and optionally a barcode, to the 3′ end of the amplified 5′- and 3′-ligated products. In some embodiments, N number of different samples of small RNAs can be characterized in N number of different reactions, wherein each reaction incorporates a different single barcode or dual barcode pair into the amplified 5′- and 3′-ligated products. The number of different barcodes can be equal to the N number of different reactions. The addition of the barcode to the 5′- and 3′-ligated product can enable the association of the particular small RNA species and reaction from which the barcoded nucleic acid sequence was derived especially when the N number of reactions are pooled and sequenced together. For example, a small RNA sequence identified by the barcode can enable the identification of the reaction associated with the barcode.

In some embodiments, the barcode sequences can comprise from about 4 to about 10 or more nucleotides. In some cases, the length of a barcode sequence can be about 4, 5, 6, 7, 8, 9, 10 nucleotides, or longer. In some cases, the length of a barcode sequence can be at least about 4, 5, 6, 7, 8, 9, 10 nucleotides, or longer. In some cases, the length of a barcode sequence can be at most about 4, 5, 6, 7, 8, 9, 10 nucleotides, or shorter.

In some embodiments, the barcodes can be 3′ single index barcodes. In some embodiments, the barcodes can be 5′/3′ dual index barcodes. In some embodiments, the barcodes are short sequences comprising 4, 5, 6, 7, or 8 nucleotides. In specific embodiments, the barcodes utilize a single indexed adapter containing a 6-nucleotide unique sequence. Commercially available kits comprising reverse barcode primers that comprise the barcodes and the 3′ sequencing adapter sequence can be used in the methods described herein. Suitable reverse barcode primers for use in the methods described herein include NEXTFLEX® barcodes sets A and B (PerkinElmer). Commercially available kits (such as NEXTFLEX Unique Dual Index (UDI) kit (barcodes 1-384) comprising dual index barcode primers with forward and reverse barcode primers containing adapter sequences can be used in the methods described herein.

In some embodiments, the preliminary library is diluted to about 10,000-50,000 molecules. In some embodiments, the preliminary library is diluted to about 10,000, about 20,000, to about 30,000, to about 40,000, or about 50,000 molecules. In some embodiments, the preliminary library is diluted to about 10,000 molecules. In some embodiments, a second amplification reaction is performed for the diluted library to generate a library of amplified 5′- and 3′-ligated products using the forward primer and the reverse barcode primer used to generate the sequencing library.

In general, the DNA polymerase is a thermostable DNA polymerase. The thermostable DNA polymerase can be recombinant and/or engineered for improved fidelity, stability, performance, etc. The thermostable DNA polymerase can be a Taq, Pfu, Pfx, Bst, Tfi, Tth DNA polymerase or derivative, modified version, or variant thereof. In some embodiments, the thermostable DNA polymerase can be a high-fidelity polymerase (e.g., Phusion polymerase; NEB). This step of synthesizing/amplifying can comprise about five cycles of denaturation, annealing, and extension. The temperature and duration of the steps can and will vary. In general, the denaturation temperature can range from about 94-98° C., the annealing temperature depends upon the melting temperature (Tm) of the primers and can range from about 48-72° C., and the extension temperature can range from about 68-72° C. The amplification step for the first amplification reaction can comprise about 3-6 amplification cycles. In specific embodiments, the amplification step for the first amplification comprises 5 amplification cycles. The amplification step for the second amplification reaction can comprise about 30-34 amplification cycles. In specific embodiments, the amplification step for the second amplification comprises 32 amplification cycles. In some embodiments, the preliminary library and/or library of 5′ and 3′ -ligated products can be purified by routine means (e.g., magnetic bead purification, spin column purification) and quantified prior to sequencing.

### Sequencing

The next step of the method comprises sequencing the library to generate sequencing fragments of forward and reverse reads of the amplified 5′- and 3′-ligated products. The amplified 5′- and 3′-ligated product comprises the UMI, the small RNA, and the at least one random nucleotide from the 3′ adapter. In some embodiments, libraries from two or more different reactions can be pooled before sequencing the libraries, wherein each library has a different single barcode or dual barcode pair incorporated into the amplified adapter-ligated products. In some embodiments, the libraries are pooled in equimolar ratios to generate a sequencing pool. In general, the sequencing method is a high throughput, massively parallel, deep sequencing method (i.e., next generation sequencing). In some embodiments, the sequencing method comprises a next generation sequencing platform. In some embodiments, the sequencing platform can be MiSeq (from Illumina), Roche 454, GS FLX Titanium, Illumina HiSeq, Illumina NextSeq, Illumina Genome analyzer IIX, Life Technologies SOLiD4, Life Technologies Ion Proton, Complete Genomics, Helicos Biosciences, Heliscope, Pacific Biosciences SMRT, or Ion Torrent PGM. As such, preparation and sequencing of the library is performed according to the manufacturer's instructions.

### Analyzing

The sequencing fragments are analyzed and processed to determine the nucleotide sequences of the plurality of the small RNAs and the relative abundance of the nucleotide sequences thereby characterizing the small RNAs in the sample. In some embodiments, paired-end sequencing fragments are merged, counted, and binned based on the UMI, sequences with the same UMI sequences are deduplicated, and the 5′ adapter sequences, which include the UMI sequences, and the 3′ adapter sequences are trimmed from the sequence fragments thereby generating corresponding nucleotide sequences of the plurality of small RNAs. In some embodiments, a fixed sequence, e.g., RYRY or ACAC, can be used to filter out sequences that have a faulty UMI. The nucleotide sequences of the plurality of small RNAs are compared or aligned to a reference small RNA sequence to identify full-length small RNA with no sequence variation relative to the reference small RNA. In some embodiments, the analyzing of the sequencing fragments further comprises identifying and quantifying sequence variants. In some embodiments, the sequence variants comprise 5′ truncated sequences, 3′ truncated sequences, sequences comprising a substitution, insertion, and/or deletion of at least one nucleotide, or a combination thereof. In some embodiments, at least about 5-1000, at least about 5-500, at least about 5-100, at least about 5-50, at least about 5-20, at least about 10-1000, at least about 10-500, at least about 10-100, at least about 10-50, at least about 10-20, at least about 15-1000, at least about 15-500, at least about 15-100, at least about 15-50, at least about 15-20, at least about 20-1000, at least about 20-500, at least about 20-100, at least about 20-50, at least about 10-30, at least about 15-25, at least about 50-1000, at least about 50-500, at least about 100-1000, at least about 100-500, at least about 200-750, or at least about 100-200 reads of the UMI are performed to generate a consensus sequence. In some embodiments, at least about 5, at least about 10, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 35, at least about 40, at least about 50, at least about 75, at least about 100, at least about 125, at least about 150, at least about 200, at least about 300, at least about 400, at least about 500, at least about 600, at least about 700, at least about 800, at least about 900, or at least about 1000 reads of the UMI are performed to generate a consensus sequence.

The raw sequencing data can be analyzed using a variety of commercial, freeware, and proprietary analysis tools such that the input or starting small RNA can be characterized. The analyzing comprises binning sequencing fragments having the same UMI sequence at the 5′ end and generating a consensus sequence corresponding to the input or starting small RNA. In embodiments in which the input or starting small RNA is a synthetic RNA, the characterizing comprises determining the relative proportion of full-length accurate sequences and/or identifying and quantifying sequence variants (e.g., 5′ truncated sequences, 3′ truncated sequences, and/or sequences comprising or more substitution, insertion, and/or deletion). In embodiments in which the input or starting small RNA is a naturally occurring RNA (e.g., miRNA, siRNA, piRNA), the characterizing comprises profiling the small RNA. Said profiling comprises characterizing sequence diversity and/or abundance (e.g., copy number).

For example, a Linux/Python-based pipeline can be used for processing raw NGS data; a Trimmomatic tool (Bolger, A. M., Lohse, M., & Usadel, B. (2014), Trimmomatic: A flexible trimmer for Illumina Sequence Data. Bioinformatics, btu170) can be used to trim degenerate bases on the 3′ end of the library; PEAR (Paired-End read merger), COPE (connecting overlapping paired end reads), FLASH (fast length adjustment of short reads, PANDAseq, or Usearch can be used to combine forward and reverse reads into a consensus sequence; AmpUMI (Clement et al., Bioinformatics, 2018, 34, i202-i210) can be used to deduplicate UMIs; and Needleman-Wunsch algorithm (e.g., the Emboss Needleall alignment tool) can be used to align the sequences with a reference sequence.

### III. SYSTEM FOR CHARACTERIZING SMALL RNA

The disclosure also provides systems for carrying out the methods described above in sections I and II. The systems comprise the 5′ adapters and 3′ adapters disclosed herein, thermocyclers, quality control electrophoresis instruments, fluorometers, spectrophotometers, incubators, mixers, sequencing machines, sequencing kits, ligation kits, PCR kits, qPCR kits, RT kits, nucleic acid purification kits, PCR plates, magnetic separators, or combinations thereof, as well as instructions for use thereof.

### IV. DEFINITIONS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

As used herein, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

As used herein, a random nucleotide (N) refers to a nucleotide that can be substituted with any of the four standard nucleotides present in DNA (e.g., A, C, G, T) or RNA (e.g., A, C, G, U). A semi-random nucleotide refers to a nucleotide that can be substitutes with two or three of the four standard nucleotides. For example, “R” refers to purines A or G, and “Y” refers to pyrimidines C or T/U.

The small RNAs referenced herein include guide RNA (gRNA), single-molecule gRNA (sgRNA), crisprRNA (crRNA), microRNA (miRNA), small interfering RNA (siRNA), Piwi-interacting (piRNA), short hairpin RNA (shRNA), and antisense RNA (asRNA).

RNA nucleobases are interchangeably referenced herein as: adenine (A or rA), cytosine (C or rC), guanine (G or rG), or uracil (U or rU).

### V. EXAMPLES

The examples below illustrate various aspects of the present disclosure.

### Example 1: Preparation of sgRNA Library and Sequencing (sgRNA-Seq)

T4 polynucleotide kinase treatment and cleanup. For each guide to be sequenced, 5 sgRNA was phosphorylated by incubating with T4 polynucleotide kinase (NEB #M0201S), polynucleotide kinase buffer, and 1 mM ATP for 30 minutes at 37° C., followed by heat inactivation at 70° C. for 2 minutes. The phosphorylated sgRNA was purified and concentrated using an RNA purification spin column kit (e.g., GeneJET RNA Purification Kit) and quantitated fluorometrically (e.g., Qubit RNA BR Assay Kit). The quality was assessed using an automated electrophoresis machine (e.g., Bioanalyzer 2100 or Tapestation 4200, Agilent).

3′ Adapter Ligations. Each guide sample was prepared in triplicate. In each well, 50 ng of input sgRNA, 1 μM of 3′ adapter (5′-rAppNNNNTGGAATTCTCGGGTGCCAAGGddC-3′; SEQ ID NO: 2), 1.25% PEG, ligase buffer, and T4 RNA ligase 2, truncated KQ (NEB #M0373S) were incubated at 25° C. for 1 hour, then 70° C. for 2 minutes, then cooled to 4° C. The 3′-ligated product was immediately purified via magnetic beads (e.g., RNAClean XP; Beckman Coulter #A63987). The final beads were resuspended in 12 μl of nuclease-free H₂O.

5′ Adapter Ligation. 10 μl of the purified 3′-ligated product was mixed with 2 μM of a 5′ adapter comprising a structured UMI (underlined sequence) (5′-rGrUrUrCrArGrArGrUrUrCrUrArCrArGrUrCrCrGrArCrGrArUrCrN₁₃rArCrArCrN-3′; SEQ ID NO: 3), 10% DMSO, 1 mM ATP, ligase buffer, and T4 RNA ligase 1 (NEB #M0204S) and the mixture was incubated at 25° C. for 1 hour, then 70° C. for 2 minutes, and then cooled to 4° C. The resulting 5′- 3′-ligated product was purified via magnetic beads (the final beads were eluted with 10 μl nuclease-free H₂O).

Reverse Transcription. To 8 μl of the purified product was added dNTP mix (0.5 mM each) and 10 μM RT oligo (5′-GCCTTGGCACCCGAGAATTCCA-3′; SEQ ID NO: 5). The mixture was incubated at 70° C. for 2 minutes, then immediately cooled on ice. After adding RNAse inhibitor (NEB #M0314L), DTT, RT buffer, and RT enzyme (e.g., Protoscript II RT; NEB #M0368L), the mixture was incubated for 1 hour at 42° C., then 2 minutes at 70° C., and then cooled to 4° C. The first strand of cDNA was purified via magnetic beads (the final beads were eluted with 8 μl of H₂O).

PCR 1. Two μl of the RT output was transferred to a plate well containing 0.5 μl of a reverse barcoded primer for multiplexing (e.g., NEXTFLEX® barcode sets A, B; PerkinElmer #NOVA-513305, -513306). If more than one sgRNA was being assayed, a different reverse barcoded primer was assigned for each different sgRNA. To each well was added a universal forward primer (5′-AATGATACGGCGACCACCGAGATCTACACGTTCAGAGTTCTACAGTCCGA-3′; SEQ ID NO: 6), dNTPs, DMSO, polymerase buffer, and thermostable DNA polymerase (e.g., Phusion DNA polymerase; NEB #M0530L). The mixture was subjected to the following program in a thermocycler: 98° C. for 30 sec; 5 cycles of 98° C., 5 sec; 58° C., 15 sec; and 72° C., 15 sec; 72° C. for 1 minute, and hold at 6° C. The PCR product was purified (SPRlselect PCR Purification & Cleanup: Beckman Coulter #B23317), and the purified cDNA was quantitated (e.g., KAPA Illumina qPCR kit; Kapa #KK4824). The sample was diluted to a concentration of about 4000 molecules/μl.

PCR 2. Five μl of sample input (20,000 molecules) was added to a well of a new plate containing 0.5 μl of a reverse barcoded primer, as noted above in PCR 1 so that the same barcode is incorporated into the PCR product. The following was added to each well: the universal forward primer (see above), dNTPs, DMSO, polymerase buffer, and thermostable DNA polymerase (e.g., Phusion DNA polymerase; NEB #M0530L). The mixture was subjected to the following program in a thermocycler: 98° C. for 30 sec; 32 cycles of 98° C., 5 sec; 58° C., 15 sec; and 72° C., 15 sec; 72° C. for 1 minute, and hold at 6° C. The PCR product was purified and quantified as described above. Each sample was normalized to 4 nM and pooled in equimolar ratios (4 nM for each) to generate a sequencing sample pool.

Sequencing. Samples were prepared for sequencing in a MiSeq benchtop sequencer (Illumina) using a MiSeq v3 600-cycle kit, per manufacturer's instructions. The sequencing read length was 101 bases in both directions, and the sequencing read depth was 300,000 to 1,000,000 reads per sample.

Data Processing. Commercial, freeware, and proprietary data analysis packages were used to process the sequencing data. For example, a Linux/Python-based pipeline was developed for processing raw NGS data; degenerate bases on the 3′ end of the library were trimmed using the Trimmomatic tool (Bolger, A. M., Lohse, M., & Usadel, B. (2014), Trimmomatic: A flexible trimmer for Illumina Sequence Data. Bioinformatics, btu170); forward and reverse reads were combined into a consensus sequence with PEAR (Paired-End read merger); UMI deduplication was performed by AmpUMI (Clement et al., Bioinformatics, 2018, 34, i202-i210); alignment against guide sequence was performed with a Needleman-Wunsch algorithm (e.g., the Emboss Needleall alignment tool); and sequences were counted and binned into annotated, unique occurrences. From this analysis, the percentage of full length, accurate sgRNA sequences was calculated, as well as the percentages of sequence variants (e.g., 5′ truncations, 3′ truncations, sequences with one mismatch, sequences with two mismatches, sequences with an insertion, sequences with a deletion, etc.).

### Example 2: Purity and Variant Profiling of sgRNAs from Different Suppliers

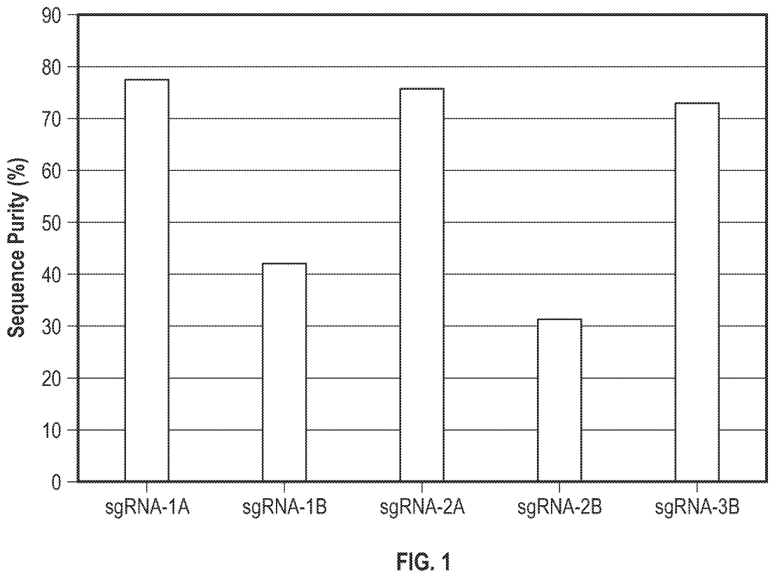

sgRNAs targeted to two different genes (sgRNA-1 and sgRNA-2) were sourced from two different suppliers (Supplier A and Supplier B). Each sgRNA was sequenced and analyzed as described above in Example 1. FIG. 1 presents the percentages of full-length accurate sequences in each sgRNA sample. This analysis revealed that the sgRNAs from Supplier A (sgRNA-1A and sgRNA-2A) had much higher purity (-71-78%) than the sgRNAs from Supplier B (-31-45%). Another sgRNA targeting a third gene was also sourced from Supplier B (gRNA-3B) and had high purity (-73%).

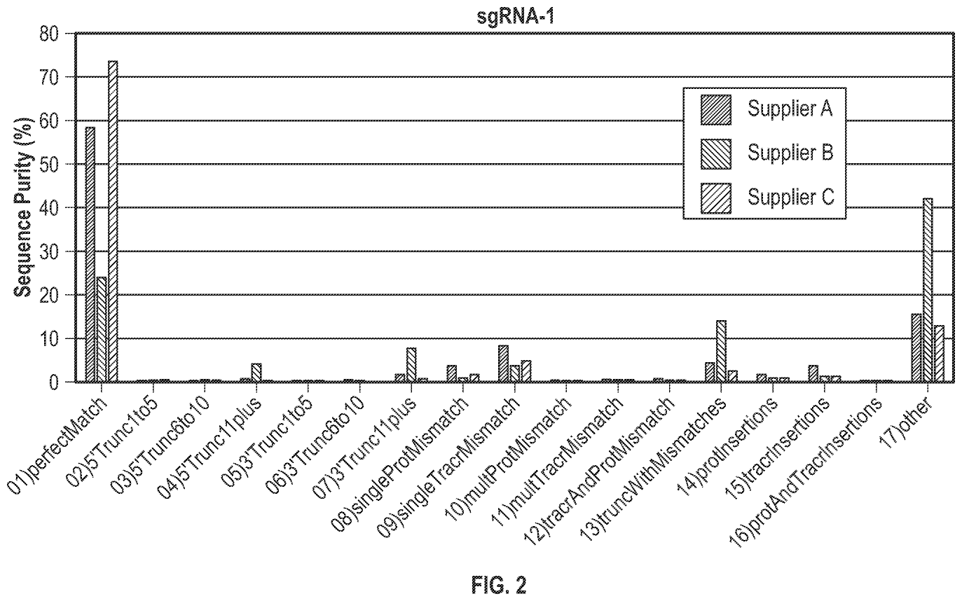

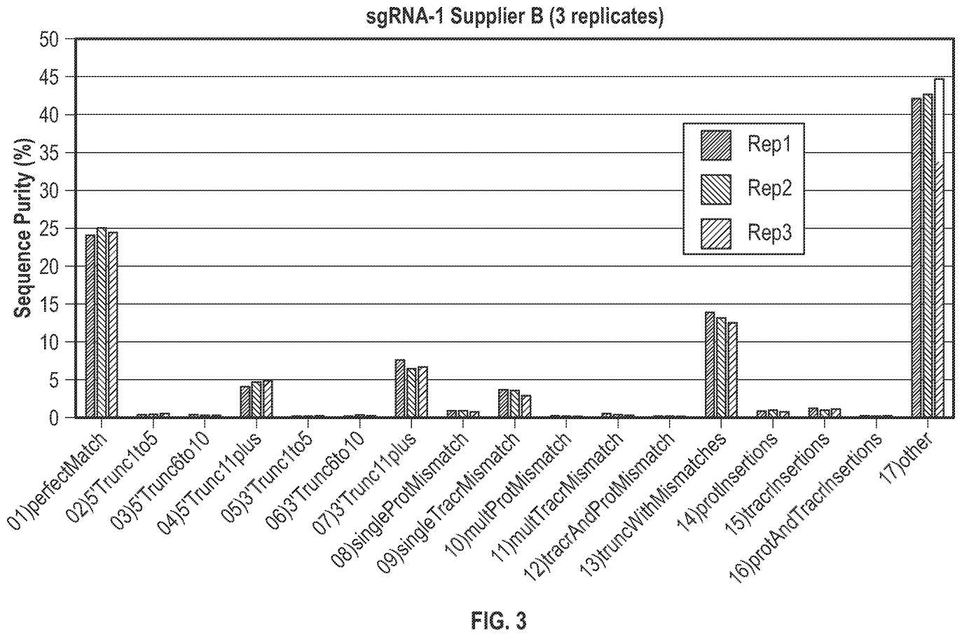

sgRNA-1 and sgRNA-2 was sourced from a third supplier (Supplier C) and compared to Supplier A and B. Each sgRNA was sequenced and analyzed as described above in Example 1. FIG. 2 presents the percentages of the most common sequence variants present in the three sources of sgRNA-1. Variant sequences include 100% match (e.g., full length), 5′ truncations—1-5 nt, 5′ truncations—6-10 nt, 5′ truncations—11+nt, 3′ truncations—1-5 nt, 3′ truncations—6-10 nt, 3′ truncations—11+nt, single protospacer mismatch, single tracr mismatch, multiple protospacer mismatches, multiple tracr mismatches, tracer and protospacer mismatches, truncation with mismatches, protospacer insertion(s), tracr insertion(s), protospacer and tracr insertions, and combinations thereof. The percentages of perfect matches ranged from about 25% from Supplier B to about 75% from Supplier C. The reproducibility of the results is presented in FIG. 3, which presents the percentages of sequence variants among three replicates of sgRNA-1 from Supplier B.

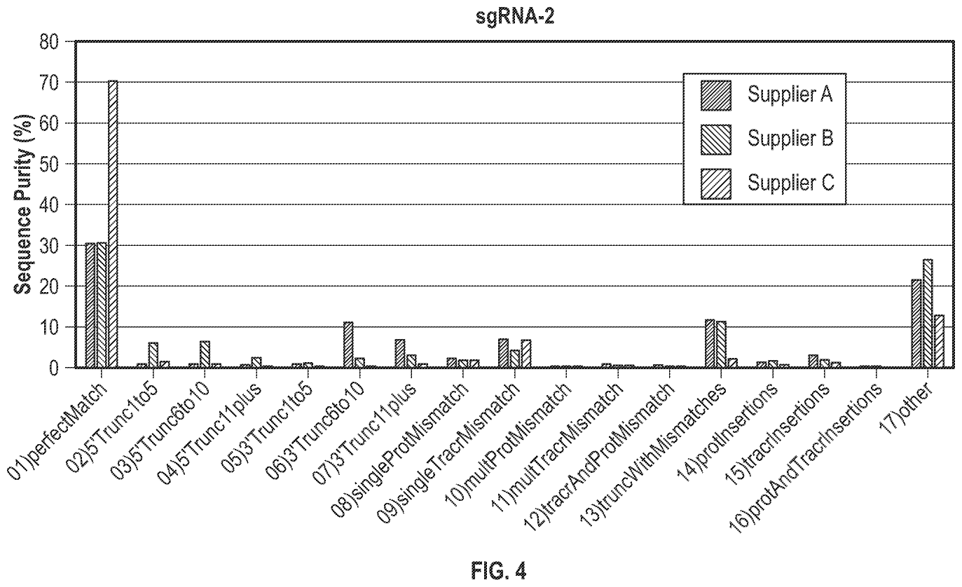

FIG. 4 presents the percentages of sequences variants in the three sources of sgRNA-2. The percentages of perfect matches ranged from about 30% from Suppliers A and B to about 70% from Supplier C.

### Example 3: Analysis of Sensitivity and Accuracy of the sgRNA-Seq Method

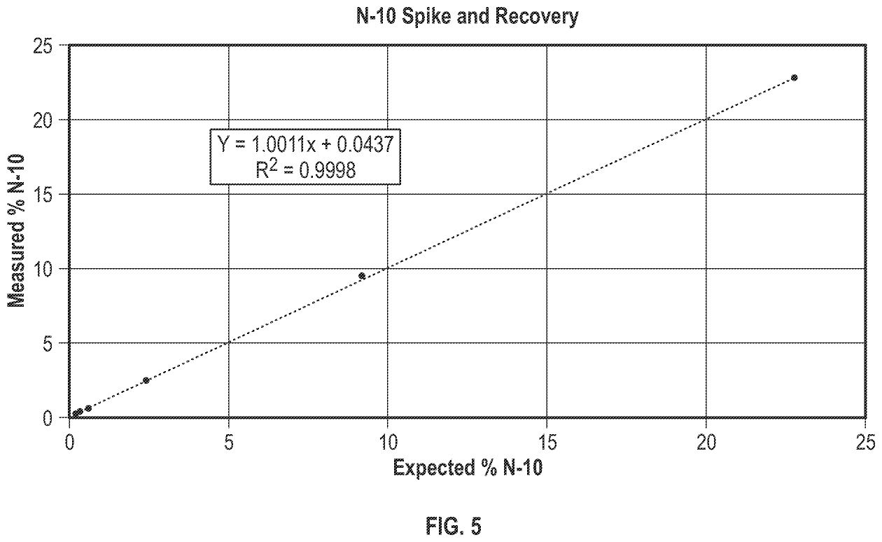

To examine the sensitivity and accuracy of the sgRNA sequencing method, varying amounts of a 5′ truncated (N-10) sgRNA targeted to a fourth gene (sgRNA-4) were spiked into full-length sgRNA-4 sgRNA. The amount of N-10 spiked into full-length sgRNA-4 ranged from 1:1 to 1:1000. FIG. 5 shows that the measured recovery was highly correlated with the expected recovery.

### Example 4: Analysis of Structured and Unstructured UMIs

sgRNAs from the two suppliers were analyzed via sgRNA-Seq using 5′ adapters comprising a standard unstructured UMI (e.g., N₁₆) or a structured UMI (e.g., N₁₃RYRYN or N₁₃CACAN). Python code was written to search for expected UMI structure, and faulty UMIs were discarded. For example, filtering for ACAC resulted in a discard rate of about 5%. Table 2 presents the percentage of full-length accurate sequences (% purity) and the percent coefficient of variance (% CV) for the different UMIs. It was found that using the ACAC structured UMI resulted in higher purity levels, as well as decreased %CV in the less pure sample.

TABLE 2
UMI Analysis

| | Unstructured UMI | | RYRY UMI | | ACAC UMI | |
|---|---|---|---|---|---|---|
| | % Purity | % CV | % Purity | % CV | % Purity | % CV |
| Supplier A | 73.93 | 2.44 | 71.62 | 2.55 | 76.63 | 2.47 |
| Supplier B | 40.38 | 14.61 | 40.19 | 12.17 | 46.66 | 1.90 |

### Example 5: Preparation of sgRNA Library and Sequencing (sgRNA-Seq)

T4 polynucleotide kinase treatment and cleanup. For each guide to be sequenced, 5 μg sgRNA was phosphorylated by incubating with T4 polynucleotide kinase (NEB #M0201S), polynucleotide kinase buffer, and 1 mM ATP for 30 minutes at 37° C., followed by heat inactivation at 70° C. for 2 minutes. The phosphorylated sgRNA was purified and concentrated using an RNA purification spin column kit (e.g., GeneJET RNA Purification Kit) and quantitated fluorometrically (e.g., Qubit RNA BR Assay Kit). The quality was assessed using an automated electrophoresis machine (e.g., Bioanalyzer 2100 or Tapestation 4200, Agilent).

3′ Adapter Ligations. Each guide sample was prepared in triplicate. In each well, 50 ng of input sgRNA, 0.3 μM of 3′ adapter (5′-rAppNNNNTGGAATTCTCGGGTGCCAAGGddC-3′; SEQ ID NO: 2), 12.5% PEG, ligase buffer, and T4 RNA ligase 2, truncated KQ (NEB #M0373S) were incubated at 25° C. for 1 hour, then 70° C. for 2 minutes, then cooled to 4° C. The 3′-ligated product was immediately purified via magnetic beads (e.g., RNAClean XP; Beckman Coulter #A63987). The final beads were resuspended in 12 μl of nuclease-free H₂O.

5′ Adapter Ligation. 10 μl of the purified 3′-ligated product was mixed with 0.3 μM of a 5′ adapter comprising a structured UMI (underlined sequence) (5′-rGrUrUrCrArGrArGrUrUrCrUrArCrArGrUrCrCrGrArCrGrArUrCrN₁₃rArCrArCrN-3′; SEQ ID NO: 3), 10% DMSO, 1 mM ATP, ligase buffer, and T4 RNA ligase 1 (NEB #M0204S) and the mixture was incubated at 25° C. for 1 hour, then 70° C. for 2 minutes, and then cooled to 4° C. The resulting 5′-3′-ligated product was purified via magnetic beads (the final beads were eluted with 10 μl nuclease-free H₂O).

Reverse Transcription. To 8 μl of the purified product was added dNTP mix (10 nmol each) and 40 fmol RT oligo (5′-GCCTTGGCACCCGAGAATTCCA-3′; SEQ ID NO: 5). The mixture was incubated at 70° C. for 2 minutes, then immediately cooled on ice. After adding 0.2 μl RNAse inhibitor (NEB #M0314L), 2 μl DTT, RT buffer, and RT enzyme (e.g., Protoscript II RT; NEB #M0368L), the mixture was incubated for 1 hour at 42° C., then 2 minutes at 70° C., and then cooled to 4° C. The first strand of cDNA was purified via magnetic beads (the final beads were eluted with 8 μl of H₂O).

PCR 1. Two μl of the RT output was transferred to a plate well containing 1 μl of 5 μM a reverse barcoded primer for multiplexing (e.g., NEXTFLEX® barcode sets A, B; PerkinElmer #NOVA-513305, -513306). If more than one sgRNA was being assayed, a different reverse barcoded primer was assigned for each different sgRNA. To each well was added a 0.25 μM universal forward primer (5′-AATGATACGGCGACCACCGAGATCTACACGTTCAGAGTTCTACAGTCCGA-3′; SEQ ID NO: 6), 0.2 mM dNTPs, 3% DMSO, polymerase buffer, and thermostable DNA polymerase (e.g., Phusion DNA polymerase; NEB #M0530L). The mixture was subjected to the following program in a thermocycler: 98° C. for 30 sec; 5 cycles of 98° C., 5 sec; 58° C., 15 sec; and 72° C., 15 sec; 72° C. for 1 minute, and hold at 6° C. The PCR product was purified (SPRlselect PCR Purification & Cleanup: Beckman Coulter #B23317), and the purified cDNA was quantitated (e.g., KAPA Illumina qPCR kit; Kapa #KK4824). The sample was diluted to a concentration of about 4000 molecules/μl.

PCR 2. Five μl of sample input (˜20,000 molecules) was added to a well of a new plate containing 1 μl of 5 μM of a reverse barcoded primer, as noted above in PCR 1 so that the same barcode is incorporated into the PCR product. The following was added to each well: 0.25 μM the universal forward primer (see above), 0.2 mM dNTPs, 3% DMSO, polymerase buffer, and thermostable DNA polymerase (e.g., Phusion DNA polymerase; NEB #M0530L). The mixture was subjected to the following program in a thermocycler: 98° C. for 30 sec; 32 cycles of 98° C., 5 sec; 58° C., 15 sec; and 72° C., 15 sec; 72° C. for 1 minute, and hold at 6° C. The PCR product was purified and quantified as described above. Each sample was normalized to 4 nM and pooled in equimolar ratios (4 nM for each) to generate a sequencing sample pool.

Sequencing. Samples were prepared for sequencing in a MiSeq benchtop sequencer (Illumina) using a MiSeq v3 600-cycle kit, per manufacturer's instructions. The sequencing read length was 101 bases in both directions, and the sequencing read depth was 300,000 to 3,000,000 reads per sample.

Data Processing. Commercial, freeware, and proprietary data analysis packages were used to process the sequencing data. For example, a Linux/Python-based pipeline was developed for processing raw NGS data; degenerate bases on the 3′ end of the library were trimmed using the Trimmomatic tool (Bolger, A. M., Lohse, M., & Usadel, B. (2014), Trimmomatic: A flexible trimmer for Illumina Sequence Data. Bioinformatics, btu170); forward and reverse reads were combined into a consensus sequence with PEAR (Paired-End read merger); UMI deduplication was performed by AmpUMI (Clement et al., Bioinformatics, 2018, 34, i202-i210); alignment against guide sequence was performed with a Needleman-Wunsch algorithm (e.g., the Emboss Needleall alignment tool); and sequences were counted and binned into annotated, unique occurrences. From this analysis, the percentage of full length, accurate sgRNA sequences was calculated, as well as the percentages of sequence variants (e.g., 5′ truncations, 3′ truncations, sequences with one mismatch, sequences with two mismatches, sequences with an insertion, sequences with a deletion, etc.).

1. A method for characterizing oligonucleotides in a sample, the method comprising:
(a) providing a sample comprising a plurality of oligonucleotides;
(b) ligating a plurality of 5′ adapters and 3′ adapters to the plurality of oligonucleotides to generate a plurality of adapter-ligated products, the 5′ adapter is ligated to the 5′ end of the oligonucleotide and the 3′ adapter is ligated to the 3′ end of the oligonucleotide, the 5′ adapter comprising a unique molecular identifier (UMI) comprising 5′-(N)₁₀₋₁₆RYRY(N)₁₋₅-3′, or optionally 5′-(N)₁₀₋₁₆ACAC(N)₁₋₅-3′, wherein N is A, C, G, or T/U, R is A or G, and Y is C or T/U, and the 3′ adapter comprising at least one random nucleotide at its 5′ end;
(c) amplifying the plurality of adapter-ligated products using a forward primer and a reverse primer to generate a library;
(d) sequencing the library to generate sequencing fragments of forward and reverse reads of the adapter-ligated products, wherein the amplified adapter-ligated product comprises the UMI, the oligonucleotide, and the 3′ adapter; and
(e) analyzing and processing the sequencing fragments to determine the nucleotide sequences of the plurality of the oligonucleotides and the relative abundance of the nucleotide sequences thereby characterizing the oligonucleotides in the sample.

2. The method of claim 1, wherein the sequencing fragments are merged, counted, and binned based on the UMI, sequences with the same UMI sequences are deduplicated, the 5′ adapter sequences, which include the UMI sequences, and the 3′ adapter sequences are trimmed from the sequencing fragments thereby generating corresponding nucleotide sequences of the plurality of oligonucleotides, and the nucleotide sequences of the plurality of oligonucleotides are compared to a reference oligonucleotide sequence to identify full-length oligonucleotides with no sequence variation relative to the reference oligonucleotide.

3. The method of claim 1 or 2, wherein the analyzing of the sequencing fragments further comprises identifying and quantifying sequence variants.

4. The method of claim 3, wherein the sequence variants comprise 5′ truncated sequences, 3′ truncated sequences, sequences comprising a substitution, insertion, and/or deletion of at least one nucleotide, or a combination thereof.

5. The method of any one of claims 1 to 4, wherein the 5′ adapter comprises 5′-(N)₁₀₋₁₆RYRY(N)₁₋₅-3′ and the sequencing fragments are filtered for the presence of the nucleotide sequence RYRY before trimming the 5′ adapter sequences, which include the UMI sequences, and the 3′ adapter sequences from the sequencing fragments.

6. The method of any one of claims 1 to 5, wherein the 5′ adapter comprises 5′-(N)₁₀₋₁₆ACAC(N)₁₋₅-3 and the sequencing fragments are filtered for the presence of the nucleotide sequence ACAC before trimming the 5′ adapter sequences, which include the UMI sequences, and the 3′ adapter sequences from the sequencing fragments.

7. The method of any one of claims 1 to 6, wherein the UMI of the 5′ adapter is 5′-N₁₃RYRYN-3′.

8. The method of any one of claims 1 to 6, wherein the UMI of the 5′ adapter is 5′-N₁₃ACACN-3′.

9. The method of any one of claims 1 to 8, wherein the 5′ adapters and 3′ adapters are ligated consecutively to the plurality of oligonucleotides.

10. The method of any one of claims 1 to 8, wherein the 3′ adapters are ligated to the plurality of oligonucleotides before the 5′ adapters.

11. The method of claim 10, wherein the plurality of oligonucleotides is phosphorylated, and optionally purified by a chromatography method, prior to ligating the 3′ adapters.

12. The method of any one of claims 1 to 11, wherein the 3′ adapter further comprises a unique sequence at its 3′ end, the unique sequence comprising a complement sequence of a portion or all of a reverse primer.

13. The method of any one of claims 1 to 12, wherein the 5′ adapter further comprises a unique sequence located 5′ of the UMI, wherein the unique sequence corresponds to a portion or all of a forward primer.

14. The method of any one of claims 1 to 13, wherein the oligonucleotides are synthetic or naturally occurring.

15. The method of any one of claims 1 to 14, wherein the oligonucleotides are gRNAs, miRNAs, siRNAs, shRNAs, RNA adapters, RNA primers, RNA probes, antisense DNAs, DNA adapters, DNA primers, or DNA probes.

16. The method of claim 15, wherein the gRNAs are sgRNAs or crRNAs.

17. The method of any one of claims 1 to 16, wherein the oligonucleotides have a length of about 30-120 nucleotides.

18. The method of any one of claims 1 to 17, wherein the oligonucleotides are RNA.

19. The method of claim 18, further comprising reverse transcribing the plurality of adapter-ligated products to generate a plurality of first strand cDNAs before step (c).

20. The method of claim 19, wherein step (c) comprises synthesizing a plurality of second strand cDNAs from the plurality of first strand cDNAs and amplifying the plurality of first strand and second strand cDNAs in a first amplifying reaction to generate a preliminary library of amplified adapter-ligated products using a forward primer and a reverse barcode primer.

21. The method of claim 20, wherein the forward primer incorporates a 5′ sequencing adapter sequence, and optionally a barcode, to the 5′ end of the amplified adapter-ligated products, and the reverse barcode primer incorporates a 3′ sequencing adapter sequence, and optionally a barcode, to the 3′ end of the amplified adapter-ligated products.

22. The method of claim 21, wherein the reverse barcode primer incorporates a barcode to the 3′ end of the amplified adapter-ligated products.

23. The method of claim 21 or 22, wherein the barcode comprises 4 to 8 nucleotides, or optionally 6 nucleotides.

24. The method of any one of claims 20 to 23, further comprising diluting the preliminary library to about 10,000-50,000 molecules and performing a second amplification reaction to generate a library of amplified adapter-ligated products using the forward primer and the reverse barcode primer.

25. The method of any one of claims 1 to 24, wherein the 3′ adapter is DNA.

26. The method of any one of claims 1 to 25, wherein the 3′ adapter is pre-adenylated at its 5′ end and dideoxy-terminated at its 3′ end.

27. The method of any one of claims 1 to 26, wherein the 3′ adapter comprises two, three, four, or five random nucleotides (N) at its 5′ end.

28. The method of any one of claims 1 to 27, wherein the 3′ adapter comprises four random nucleotides (N) at its 5′ end.

29. The method of any one of claims 1 to 28, wherein the 3′ adapter comprises 5′-NNNNTGGAATTCTCGGGTGCCAAGGddC-3′ (SEQ ID NO: 1) or 5′-rAppNNNNTGGAATTCTCGGGTGCCAAGGddC-3′ (SEQ ID NO: 2).

30. The method of any one of claims 1 to 29, wherein the 5′ adapter is RNA and the UMI comprises 5′-(rN)₁₀₋₁₆rRrYrRrY(rN)₁₋₅-3′, or optionally 5′-(rN)₁₀₋₁₆rArCrArC(rN)₁₋₅-3′, wherein rN is rA, rC, rG, or rU, rR is rA or rG, and rY is rC or rU.

31. The method of claim 30, wherein the UMI of the 5′ adapter is 5′-rN₁₃rRrYrRrYrN-3′.

32. The method of claim 30, wherein the UMI of the 5′ adapter is 5′-rNi₃rArCrArCrN-3′.

33. The method of any one of claims 30 to 32, wherein the 5′ adapter comprises 5′-rGrUrUrCrArGrArGrUrUrCrUrArCrArGrUrCrCrGrArCrGrArUrCrN₁₃rArCrArCrN-3′ (SEQ ID NO: 3).

34. The method of any one of claims 1 to 33, wherein the sequencing is a deep sequencing method.

35. A method for characterizing small RNA in a sample, the method comprising:
(a) providing a sample comprising a plurality of small RNAs;
(b) ligating a plurality of 3′ adapters to the plurality of small RNAs to generate a plurality of 3′-ligated products, the 3′ adapter is ligated to the 3′ end of the small RNA, the 3′ adapter comprising at least one random nucleotide at its 5′ end and a unique sequence at its 3′ end, the unique sequence comprising a complement sequence of a portion or all of a reverse primer;
(c) ligating a plurality of 5′ adapters to the plurality of 3′-ligated products to generate a plurality of 5′- and 3′-ligated products, the 5′ adapter is ligated to the 5′ end of the small RNA, the 5′ adapter comprising a unique molecular identifier (UMI) and a unique sequence located 5′ of the UMI, the UMI comprising 5′-(N)₁₀₋₁₆RYRY(N)₁₋₅-3′, or optionally 5′-(N)₁₀₋₁₆ACAC(N)₁₋₅-3′, wherein N is A, C, G, or T/U, R is A or G, and Y is C or T/U, and the unique sequence corresponding to a portion or all of a forward primer;
(d) reverse transcribing the plurality of 5′- and 3′-ligated products with the reverse primer to generate a plurality of first strand cDNAs;
(e) synthesizing a plurality of second strand cDNAs from the plurality of first strand cDNAs, optionally concurrently with step (f);
(f) amplifying the plurality of first strand and second strand cDNAs in a first amplifying reaction to generate a preliminary library of amplified 5′- and 3′-ligated products using a forward primer and a reverse barcode primer, the forward primer incorporating a 5′ sequencing adapter sequence, and optionally a barcode, to the 5′ end of the amplified 5′- and 3′-ligated products, the reverse barcode primer incorporating a 3′ sequencing adapter sequence, and optionally a barcode, to the 3′ end of the amplified 5′- and 3′-ligated products;
(g) diluting the preliminary library to about 10,000-50,000 molecules and performing a second amplification reaction to generate a library of amplified 5′- and 3′-ligated products using the forward primer and the reverse barcode primer;
(h) sequencing the library to generate sequencing fragments of forward and reverse reads of the amplified 5′- and 3′-ligated products, wherein the amplified 5′- and 3′-ligated product comprises the UMI, the small RNA, and the at least one random nucleotide; and
(i) analyzing and processing the sequencing fragments to determine the nucleotide sequences of the plurality of the small RNAs and the relative abundance of the nucleotide sequences thereby characterizing the small RNAs in the sample.

36. The method of claim 35, wherein the sequencing fragments are merged, counted, and binned based on the UMI, sequences with the same UMI sequences are deduplicated, the 5′ adapter sequences, which include the UMI sequences, and the 3′ adapter sequences are trimmed from the sequence fragments thereby generating corresponding nucleotide sequences of the plurality of small RNAs, and the nucleotide sequences of the plurality of small RNAs are compared to a reference small RNA sequence to identify full-length small RNA with no sequence variation relative to the reference small RNA.

37. The method of claim 35 or 36, wherein the analyzing of the sequencing fragments further comprises identifying and quantifying sequence variants.

38. The method of claim 37, wherein the sequence variants comprise 5′ truncated sequences, 3′ truncated sequences, sequences comprising a substitution, insertion, and/or deletion of at least one nucleotide, or a combination thereof.

39. The method of any one of claims 35 to 38, wherein the 5′ adapter comprises 5′-(N)₁₀₋₁₆RYRY(N)₁₋₅-3′ and the sequencing fragments are filtered for the presence of the nucleotide sequence RYRY before trimming the 5′ adapter sequences, which include the UMI sequences, and the 3′ adapter sequences from the sequencing fragments.

40. The method of any one of claims 35 to 39, wherein the 5′ adapter comprises 5′-(N)₁₀₋₁₆ACACN-3 and the sequencing fragments are filtered for the presence of the nucleotide sequence ACAC before trimming the 5′ adapter sequences, which include the UMI sequences, and the 3′ adapter sequences from the sequencing fragments.

41. The method of any one of claims 35 to 40, wherein the small RNA is a synthetic RNA, the synthetic RNA being a gRNA, a siRNA, a shRNA, an RNA adapter, an RNA primer, or an RNA probe.

42. The method of claim 41, wherein the gRNA is a sgRNA or a crRNA.

43. The method of any one of claims 35 to 40, wherein the small RNA is a naturally occurring RNA, the naturally occurring RNA being a miRNA, a siRNA, or a piRNA.

44. The method of any one of claims 35 to 43, wherein the small RNA has a length from about 30-120 nucleotides.

45. The method of any one of claims 35 to 44, wherein the small RNA is phosphorylated, and optionally purified by a chromatography method, prior to step (b).

46. The method of any one of claims 35 to 45, wherein the 3′ adapter is DNA.

47. The method of any one of claims 35 to 46, wherein the 3′ adapter is preadenylated at its 5′ end and dideoxy-terminated at its 3′ end.

48. The method of any one of claims 35 to 47, wherein the 3′ adapter comprises two, three, four, or five random nucleotides (N) at its 5′ end, wherein N is A, C, G, or T.

49. The method of any one of claims 35 to 48, wherein the 3′ adapter comprises four random nucleotides (N) at its 5′ end.

50. The method of any one of claims 35 to 49, wherein the 3′ adapter comprises 5′-NNNNTGGAATTCTCGGGTGCCAAGGddC-3′ (SEQ ID NO: 1) or 5′-rAppNNNNTGGAATTCTCGGGTGCCAAGGddC-3′ (SEQ ID NO: 2).

51. The method of any one of claims 35 to 50, wherein the ligating in step (b) comprises contact with a T4 RNA ligase 2.

52. The method of any one of claims 35 to 51, wherein the 3′-ligated product is purified using magnetic beads prior to step (c).

53. The method of any one of claims 35 to 52, wherein the 5′ adapter is RNA and the UMI comprises 5′-(rN)₁₀₋₁₆rRrYrRrY(rN)₁₋₅-3′, or optionally 5′-(rN)₁₀₋₁₆rArCrArC(rN)₁₋₅-3′, wherein rN is rA, rC, rG, or rU, rR is rA or rG, and rY is rC or rU and r signifies an RNA base.

54. The method of claim 53, wherein the UMI of the 5′ adapter is 5′-rN₁₃rRrYrRrYrN-3′.

55. The method of claim 53, wherein the UMI of the 5′ adapter is 5′-rN₁₃rArCrArCrN-3′.

56. The method of any one of claims 53 to 55, wherein the 5′ adapter comprises 5′-rGrUrUrCrArGrArGrUrUrCrUrArCrArGrUrCrCrGrArCrGrArUrCrN₁₃rArCrArCrN-3′ (SEQ ID NO: 3).

57. The method of any one of claims 35 to 56, wherein the ligating in step (c) comprises contact with a T4 RNA ligase 1.

58. The method of any one of claims 35 to 57, wherein the 5′- and 3′-ligated product is purified using magnetic beads prior to step (d).

59. The method of any one of claims 35 to 58, wherein the first strand cDNA is purified using magnetic beads prior to step (e).

60. The method of any one of claims 35 to 59, wherein the reverse barcode primer incorporates a barcode to the 3′ end of the amplified adapter-ligated products.

61. The method of any one of claims 35 to 60, wherein the barcode comprises 4 to 8 nucleotides, or optionally comprise 6 nucleotides.

62. The method of any one of claims 35 to 61, wherein step (f) comprises about 3-6 amplification cycles, or optionally 5 amplification cycles.

63. The method of any one of claims 35 to 62, wherein the cDNA from step (f) is purified using magnetic beads prior to (g).

64. The method of any one of claims 35 to 63, wherein step (g) comprises about 30-34 amplification cycles, or optionally 32 amplification cycles.

65. The method of any one of claims 35 to 64, wherein the library is purified using magnetic beads prior to step (h).

66. The method of any one of claims 35 to 65, wherein the sequencing is a deep sequencing method.


SEQUENCE LISTING (6 sequences)

SEQ ID NO: 1 - DNA, 26 bp, synthetic construct
  source 1..26: mol_type = other DNA
  misc_feature 26: note = 3' dideoxy-termination
nnnntggaattctcgggtgccaaggc

SEQ ID NO: 2 - DNA, 26 bp, synthetic construct
  source 1..26: mol_type = other DNA
  misc_feature 1: note = 5'-pre-adenylation
  misc_feature 26: note = 3' dideoxy-termination
nnnntggaattctcgggtgccaaggc

SEQ ID NO: 3 - RNA, 43 nt, synthetic construct
  source 1..43: mol_type = other RNA
gttcagagttctacagtccgacgatcnnnnnnnnnnnnnacac

SEQ ID NO: 4 - DNA, 43 bp, synthetic construct
  source 1..43: mol_type = other DNA
gttcagagttctacagtccgacgatcnnnnnnnnnnnnnacac

SEQ ID NO: 5 - DNA, 22 bp, synthetic construct
  source 1..22: mol_type = other DNA
gccttggcacccgagaattcca

SEQ ID NO: 6 - DNA, 50 bp, synthetic construct
  source 1..50: mol_type = other DNA
aatgatacggcgaccaccgagatctacacgttcagagttctacagtccga